(19) United States
(12) Patent Application Publication
(10) Pub. No.: US 2026/0152472 A1
(43) Pub. Date: Jun. 4, 2026

(54) Phenyl Amino Pyrimidine Compounds and Uses Thereof

(71) Applicant: GlaxoSmithKline LLC, Wilmington, DE (US)

(72) Inventors: Christopher John Burns, Caulfield North (AU); Andrew Craig Donohue, Bentleigh East (AU); John Thomas Feutrill, Rosanna (AU); Thao Lien Thi Nguyen, Bundoora (AU); Andrew Frederick Wilks, South Yarra (AU); Jun Zeng, Scoresby (AU)

(73) Assignee: GlaxoSmithKline LLC, Wilmington, DE (US)

(21) Appl. No.: 19/459,164

(22) Filed: Jan. 26, 2026

Related U.S. Application Data
(63) Continuation of application No. 17/724,825, filed on Apr. 20, 2022, now Pat. No. 12,570,614.
Application No. 17/724,825 is a continuation of application No. 14/960,710, filed on Dec. 7, 2015, now abandoned.
Application No. 14/960,710 is a division of application No. 13/913,362, filed on Jun. 7, 2013, now Pat. No. 9,238,628.
Application No. 13/913,362 is a continuation of application No. 12/530,610, now Pat. No. 8,486,941.
(60) Provisional application No. 60/894,264, filed on Mar. 12, 2007.
(60) Provisional application No. 61/016,252, filed on Dec. 21, 2007.

(51) Int. Cl. C07D 239/42; A61K 31/5377; A61P 35/00; A61P 35/02; C07D 295/02; C07D 295/12; C07D 401/12; C07D 401/14; C07D 403/12; C07D 405/12; C07D 417/12

(52) U.S. Cl. CPC C07D 239/42; A61K 31/5377; A61P 35/00; A61P 35/02; C07D 295/02; C07D 295/12; C07D 401/12; C07D 401/14; C07D 403/12; C07D 405/12; C07D 417/12

(57) ABSTRACT

The present invention relates to phenyl amino pyrimidine compounds which are inhibitors of protein kinases including JAK kinases. In particular the compounds are selective for JAK2 kinases. The kinase inhibitors can be used in the treatment of kinase associated diseases such as immunological and inflammatory diseases including organ transplants; hyperproliferative diseases including cancer and myeloproliferative diseases; viral diseases; metabolic diseases; and vascular diseases.

### CROSS-REFERENCE TO RELATED APPLICATION

This application is a continuation of U.S. patent application Ser. No. 17/724,825, filed Apr. 20, 2022, which is a continuation of U.S. patent application Ser. No. 14/960,710, filed Dec. 7, 2015, which is a divisional of U.S. patent application Ser. No. 13/913,362, filed Jun. 7, 2013, now U.S. Pat. No. 9,238,628, which is a continuation of U.S. patent application Ser. No. 12/530,610, filed Mar. 19, 2010, now U.S. Pat. No. 8,486,941, which is the national phase of International Patent Application No. PCT/AU2008/000339 having an international filing date of Mar. 12, 2008, and claims priority from U.S. Provisional Patent Application No. 60/894,264 filed Mar. 12, 2007, and U.S. Provisional Patent Application No. 61/016,252 filed Dec. 21, 2007, the contents of each of which are incorporated herein by reference in their entirety.

### SEQUENCE LISTING

The present application is being filed along with a Sequence Listing in electronic format. The Sequence Listing is provided as a file entitled SeqList-SERYM017US09.xml, created Jan. 22, 2026, which is 24,672 bytes in size. The information in the electronic format of the sequence listing is incorporated herein by reference in its entirety.

### TECHNICAL FIELD

The present invention relates to phenyl amino pyrimidine compounds which are inhibitors of protein kinases, including JAK kinases. In particular the compounds are selective for JAK2 kinases. The kinase inhibitors can be used in the treatment of kinase associated diseases such as immunological and inflammatory diseases including organ transplants; hyperproliferative diseases including cancer and myeloproliferative diseases; viral diseases; metabolic diseases; and vascular diseases.

### BACKGROUND ART

JAKs are kinases which phosphorylate a group of proteins called Signal Transduction and Activators of Transcription or STATs. When phosphorylated, STATs dimerize, translocate to the nucleus and activate expression of genes which lead to, amongst other things, cellular proliferation.

The central role played by the JAK family of protein tyrosine kinases in the cytokine dependent regulation of both proliferation and end function of several important cell types indicates that agents capable of inhibiting the JAK kinases are useful in the prevention and chemotherapeutic treatment of disease states dependent on these enzymes. Potent and specific inhibitors of each of the currently known four JAK family members will provide a means of inhibiting the action of the cytokines that drive immunological and inflammatory diseases.

Myeloproliferative disorders (MPD) include, among others, polycythemia vera (PV), primary myelofibrosis, thrombocythemia, essential thrombocythemia (ET), idiopathic myelofibrosis (IMF), chronic myelogenous leukemia (CML), systemic mastocystosis (SM), chronic neutrophilic leukemia (CNL), myelodisplastic syndrome (MDS) and systemic mast cell disease (SMCD). JAK2 is a member of the JAK family of kinases in which a specific mutation (JAK2V617F) has been found in 99% of polycythemia vera (PV) patients and 50% of essential thrombocytopenia (ET) and idiopathic myelofibrosis (MF). This mutation is thought to activate JAK2, giving weight to the proposition that a JAK2 inhibitor will be useful in treating these types of diseases.

Asthma is a complex disorder characterized by local and systemic allergic inflammation and reversible airway obstruction. Asthma symptoms, especially shortness of breath, are a consequence to airway obstruction, and death is almost invariably due to asphyxiation. Airway Hyper Responsiveness (AHR), and mucus hyper secretion by goblet cells are two of the principle causes of airway obstruction in asthma patients. Intriguingly recent work in animal experimental models of asthma has underscored the importance of IL-13 as a key player in the pathology of asthma. Using a specific IL-13 blocker, it has been demonstrated that IL-13 acts independently of IL-4 and may be capable of inducing the entire allergies asthma phenotype, without the induction of IgE (i.e., in a non-atopic fashion). This and other models have pointed to an important second tier mechanism for elicitating the pathophysiology of asthma, that is not dependent on the production of IgE by resident B-cells or the presence of conisophils. A direct induction of AHR by IL-13, represents an important process that is likely to be an excellent target for intervention by new therapies. A contemplated effect of a JAK2 inhibitor to the lungs would result in the suppression of the local release of IL-13 mediated IgE production, and therefore reduction in histaminine release by mast cells and eosinophils. This and other consequences of the absence of IL-13 indicate that many of the effects of asthma may be alleviated through administration of a JAK2 inhibitor to the lungs.

Chronic Obstructive Pulmonary Disease (COPD) is a term which refers to a large group of lung diseases which can interfere with normal breathing. Current clinical guidelines define COPD as a disease state characterized by airflow limitation which is not fully reversible. The airflow limitation is usually both progressive and associated with an abnormal inflammatory response of the lungs to noxious particles and gases, particularly cigarette smoke and pollution. Several studies have pointed to an association between increased production of IL-13 and COPD, lending support to the proposition that the potential alleviation of asthma symptoms by use of a JAK2 inhibitor, may also be achieved in COPD. COPD patients have a variety of symptoms including cough, shortness of breath, and excessive production of sputum. COPD includes several clinical respiratory syndromes including chronic bronchitis and emphysema.

Chronic bronchitis is a long standing inflammation of the bronchi which causes increased production of mucus and other changes. The patient's symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath.

Additionally, there is evidence of STAT activation in malignant tumors, among them lung, breast, colon, ovarian, prostate and liver cancer, as well as Hodgkin's lymphoma, multiple myeloma and hepatocellular carcinoma. Chromosomal translocations involving JAK2 fusions to Tcl, Bcr and PCM1 have been described in a number of hematopoietic malignancies including chronic myelogenous leukemia (CML), acute myelogenous leukemia (AML), chronic eosinophilic leukemia (CEL), myelodisplastie syndrome (MDS), myeloproliferative disease (MPD) and acute lymphocytic leukemia (ALL). This suggests treatment of hyperproliferative disorders such as cancers including multiple myeloma; prostate, breast and lung cancer; Hodgkin's Lymphoma; CML; AML; CEL; MDS; ALL; B-cell Chronic Lymphocytic Leukemia; metastatic melanoma; glioma; and hepatoma, by JAK inhibitors is indicated.

Potent inhibitors of JAK2, in addition to the above, will also be useful in vascular disease such as hypertension, hypertrophy, cardiac ischemia, heart failure (including systolic heart failure and diastolic heart failure), migraine and related cerebrovascular disorders, stroke, Raynaud's phenomenon. POEMS syndrome, Prinzmetal's angina, vasculitides, such as Takayasu's arteritis and Wegener's granulomatosis, peripheral arterial disease, heart disease and pulmonary arterial hypertension.

Pulmonary arterial hypertension (PAH) is a pulmonary vascular disease affecting the pulmonary arterioles resulting in an elevation in pulmonary artery pressure and pulmonary vascular resistance but with normal or only mildly elevated left-sided filling pressures. PAH is caused by a constellation of diseases that affect the pulmonary vasculature. PAH can be caused by or associated with collagen vascular disorders such as systemic sclerosis (scleroderma), uncorrected congenital heart disease, liver disease, portal hypertension, HIV infection, Hepatitis C, certain toxins, splenectomy, hereditary hemorrhagic teleangiectasia, and primary genetic abnormalities. In particular, a mutation in the bone morphogenetic protein type 2 receptor (a TGF-b receptor) has been identified as a cause of familial primary pulmonary hypertension (PPH). It is estimated that 6% of cases of PPH are familial, and that the rest are “sporadic.” The incidence of PPH is estimated to be approximately 1 case per 1 million population. Secondary causes of PAH have a much higher incidence. The pathologic signature of PAH is the plexiform lesion of the lung which consists of obliterative endothelial cell proliferation and vascular smooth muscle cell hypertrophy in small precapillary pulmonary arterioles. PAH is a progressive disease associated with a high mortality. Patients with PAH may develop right ventricular (RV) failure. The extent of RV failure predicts outcome. The JAK/STAT pathway has recently been implicated in the pathophysiology of PAH. JAKs are kinases which phosphorylate a group of proteins called Signal Transduction and Activators of Transcription or STATs. When phosphorylated, STATs dimerize, translocate to the nucleus and activate expression of genes which lead to proliferation of endothelial cells and smooth muscle cells, and cause hypertrophy of cardiac myocytes. There are three different isoforms of JAK: JAK1, JAK2, and JAK3. Another protein with high homology to JAKs is designated TYK2. An emerging body of data has shown that the phosphorylation of STAT3, a substrate for JAK2, is increased in animal models of PAH. In the rat monocrotaline model, there was increased phosphorylation of the promitogenic transcription factor STAT3. In this same study pulmonary arterial endothelial cells (PAECs) treated with monocrotaline developed hyperactivation of STAT3. A promitogenic agent or protein is an agent or protein that induces or contributes to the induction of cellular proliferation. Therefore, one effect of JAK2 inhibition would be to decrease proliferation of endothelial cells or other cells, such as smooth muscle cells. A contemplated effect of a JAK2 inhibitor would be to decrease the proliferation of endothelial cells or other cells which obstruct the pulmonary arteriolar lumen. By decreasing the obstructive proliferation of cells, a JAK2 inhibitor could be an effective treatment of PAH.

Additionally the use of JAK kinase inhibitors for the treatment of viral diseases and metabolic diseases is indicated.

Although the other members of the JAK family are expressed by essentially all tissues, JAK3 expression appears to be limited to hematopoetic cells. This is consistent with its essential role in signalling through the receptors for IL-2, IL4, IL-7, IL-9 and IL-15 by non-covalent association of JAK3 with the gamma chain common to these multichain receptors. Males with X-linked severe combined immunodeficiency (XSCID) have defects in the common cytokine receptor gamma chain (gamma c) gene that encodes a shared, essential component of the receptors of interleukin-2 (II-2), IL-4, IL-7, IL-9, and IL-15. An XSCID syndrome in which patients with either mutated or severely reduced levels of JAK3 protein has been identified, suggesting that immunosuppression should result from blocking signalling through the JAK3 pathway. Gene Knock out studies in mice have suggested that JAK3 not only plays a critical role in B and T lymphocyte maturation, but that JAK3 is constitutively required to maintain T cell function. Taken together with the biochemical evidence for the involvement of JAK3 in signalling events downstream of the IL-2 and IL-4 receptor, these human and mouse mutation studies suggest that modulation of immune activity through the inhibition of JAK3 could prove useful in the treatment of T-cell and B-cell proliferative disorders such as transplant rejection and autoimmune diseases. Conversely undesired inhibition of JAK3 could have a devastating effect on the immune status of an individual treated with drug.

Although the inhibition of various types of protein kinases, targeting a range of disease states, is clearly beneficial, it has been to date demonstrated that the identification of a compound which is selective for a protein kinase of interest, and has good “drug like” properties such as high oral bioavailability, is a challenging goal. In addition, it is well established that the predictability of inhibition, or selectivity, in the development of kinase inhibitors is quite low, regardless of the level sequence similarity between the enzymes being targeted.

The challenges in developing therapeutically appropriate JAK2 inhibitors for use in treatment kinase associated diseases such as immunological and inflammatory diseases including organ transplants; hyperproliferative diseases including cancer and myeloproliferative diseases; viral diseases; metabolic diseases; and vascular diseases include designing a compound with appropriate specificity which also has good drug-likeliness.

There is therefore a continuing need to design and/or identify compounds which specifically inhibit the JAK family of kinases, and particularly compounds which may preferentially inhibit one of the JAK kinases relative to the other JAK kinases, particularly JAK2. There is a need for such compounds for the treatment of a range of diseases.

### DISCLOSURE OF THE INVENTION

In a first aspect, there is provided a compound of formula I

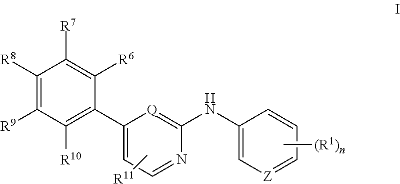

wherein
Q and Z are independently selected from N and CR¹;
n is 1, 2 or 3;
R¹ is independently selected from hydrogen, halogen, R², OR², OH, R⁴, OR⁴, CN, CF₃, (CH₂)ₙN(R²)², NO₂, R²R⁴, SO₂R⁴, NR²SO₂R³, COR⁴, NR²COR³, CO₂H, CO₂R², NR²COR⁴, R²CN, R²CN, R²OH, R²OR³ and OR⁵R⁴; or
two R¹ substituents together with the carbons which they are attached to form an unsaturated 5 or 6 membered heterocyclyl;
R² is substituted or unsubstituted C₁₋₄alkyl or substituted or unsubstituted C₁₋₄ alkylene where up to 2 carbon atoms can be optionally replaced with CO, NR^{Y}, CONR^{Y}, S, SO₂ or O;
R³ is R², C₂₋₄alkenyl or substituted or unsubstituted aryl;
R⁴ is NH₂, NHR², N(R¹)₂, substituted or unsubstituted morpholino, substituted or unsubstituted thiomorpholino, substituted or unsubstituted thiomorpholino-1-oxide, substituted or unsubstituted thiomorpholino-1,1-dioxide, substituted or unsubstituted piperazinyl, substituted or unsubstituted piperidinyl, substituted or unsubstituted pyridinyl, substituted or unsubstituted pyrrolidinyl, substituted or unsubstituted pyrrolyl, substituted or unsubstituted oxazolyl, substituted or unsubstituted imidazolyl, substituted or unsubstituted tetrahydrofuranyl and substituted or unsubstituted tetrahydropyranyl;
R⁵ is substituted or unsubstituted C₁₋₄alkylene;
R⁶-R¹⁰ are independently selected from H, R^{X}CN, halogen, substituted or unsubstituted C₁₋₄alkyl, OR¹, CO₂R¹, N(R¹)₂, NO₂, CON(R¹)₂, SO₂N(R^{Y})₂, N(SO₂R¹)₂, substituted or unsubstituted piperazinyl, N(R^{Y})SO₂R² and CF₃;
R^{x} is absent or substituted or unsubstituted C₁₋₆alkylene wherein up to 2 carbon atoms can be optionally replaced with CO, NSO₂R¹, NR^{Y}, CONR^{Y}, S, SO₂ or O;
R^{Y} is H or substituted or unsubstituted C₁₋₄alkyl; and
R¹¹ is selected from H, halogen, substituted or unsubstituted C₁₋₄ alkyl, OR², CO₂R², CN, CON(R¹)₂ and CF₃,
or an enantiomer thereof, a prodrug thereof or a pharmaceutically acceptable salt thereof.

In a second aspect, there is provided a process for the preparation of the compound of formula I defined above which comprises the step of coupling a compound of formula II

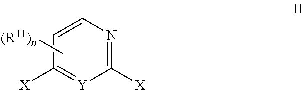

wherein
Y and R¹¹ and n are as defined above and X is a leaving group with compounds of formulae III and IV

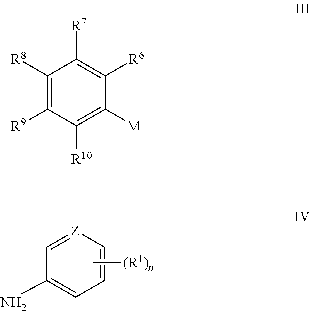

wherein
n, Z, R¹ and R⁶-R¹⁰ are as defined above; and
M is B or a metal such as Sn, Zn or Mg.

The compounds of formula I are kinase inhibitors, preferably JAK inhibitors, more preferably JAK2 inhibitors. These compounds are useful in the treatment of kinase associated diseases such as immunological and inflammatory diseases including organ transplants; hyperproliferative diseases including cancer and myeloproliferative diseases; viral diseases; metabolic diseases; and vascular diseases.

In a third aspect, there is provided a pharmaceutical agent or metabolites thereof comprising the compound of formula I defined above.

There is also provided use of the compound of formula I as a pharmaceutical agent or metabolites thereof.

There is further provided the compound of formula I defined above for use as a pharmaceutical agent or metabolites thereof.

In a fourth aspect, there is provided a kinase inhibitor comprising the compound formula I defined above.

There is also provided use of the compound of formula I defined above as a kinase inhibitor.

There is further provided the compound of formula I defined above for use as a kinase inhibitor.

In a fifth aspect, there is provided a compound of formula I defined above for use as a pharmaceutical agent or metabolites thereof, preferably a kinase inhibitor, more preferably a JAK kinase inhibitor, most preferably a JAK2 selective inhibitor.

The compound of formula I may also be administered in the form of a pharmaceutical composition together with a pharmaceutically acceptable carrier.

In a sixth aspect, there is provided a pharmaceutical composition comprising the compound of formula I defined above and a pharmaceutically acceptable carrier.

In one embodiment, the pharmaceutical composition also comprises one or more additional therapeutic agents.

The compound of formula I may be contained within or attached to an implant, such as a drug eluting stent. For example, when the compound is used for the treatment of pulmonary arterial hypertension (PAH), the compound may be contained within or attached to a pulmonary artery stent, which may act locally, or be released from the stent into the pulmonary circulation where the compound exerts its therapeutic activity in the pulmonary vasculature.

In a seventh aspect, there is provided an implant which comprises the compound of formula I defined above.

In an eighth aspect, there is provided a method for the treatment of kinase associated diseases such as immunological and inflammatory diseases including organ transplants; hyperproliferative diseases including cancer and myeloproliferative diseases; viral diseases; metabolic diseases; and vascular diseases which comprises administering an effective amount of the compound of formula I or a pharmaceutical composition defined above to a subject in need thereof.

There is also provided use of the compound of formula I or a pharmaceutical composition as defined above in the manufacture of a medicament for the treatment of kinase associated diseases such as immunological and inflammatory diseases including organ transplants; hyperproliferative diseases including cancer and myeloproliferative diseases; viral diseases; metabolic diseases; and vascular diseases.

There is further provided use of the compound of formula I or a pharmaceutical composition as defined above in the treatment of kinase associated diseases such as immunological and inflammatory diseases including organ transplants; hyperproliferative diseases including cancer and myeloproliferative diseases; viral diseases; metabolic diseases; and vascular diseases.

There is still further provided the compound of the formula I or a pharmaceutical composition defined above for use in the treatment of kinase associated diseases such as immunological and inflammatory diseases including organ transplants; hyperproliferative diseases including cancer and myeloproliferative diseases; viral diseases; metabolic diseases; and vascular diseases.

In a ninth aspect, there is provided a method of inhibiting a kinase in a cell comprising contacting the cell with the compound of formula I defined above.

### BRIEF DESCRIPTION OF THE DRAWINGS

FIG. 1 shows the amino acid sequence alignment of selected JAK Kinases. The sequences shown are j2h=JAK2 (SEQ ID NO:1), j1h=JAK1 (SEQ ID NO:2), j3h=JAK3 (SEQ ID NO: 3), and tyk2=TYK2 (SEQ ID NO:4). The sequences are numbered with position I starting at amino acid 833 of the JAK2 sequence (taken from Genbank sequence NP_004963, SEQ ID NO:5) and ends at the C-terminal amino acid. The sequences shown correspond to the C-terminal kinase domain.

FIG. 2 shows a flow cytometry analysis of STAT5 phosphorylation in untreated erythroleukemic cells (HEL 92.1.7) versus cells that have been treated with 0.25, 0.5, 1, or 2 μM Compound 3, or DMSO/STAT5py. After treatment the cells were stained with mouse monoclonal anti-STAT5(Y694) PE antibody and analyzed using fluorescence activated cell sorting (FACS). The histograms are shaded according to the fold change in median fluorescence relative to the isotype control (Isocont lane in clear outline).

FIG. 3 shows the effect of compound 3 on IL-3 induced STAT5 phosphorylation in BaF3 cells. BaF3 cells were incubated with vehicle only, increasing concentrations of compound 3 or a positive control compound. The Western blots were treated with a STAT5 phospho-specific antibody and exposed to film for 5 minutes (top blot) and 1 minute (middle blot). The bottom blot shows total STAT protein, regardless of phosphorylation state. These blots clearly show a decrease in STAT5 phosphorylation in IL-3 stimulated BaF3 Cells with increased concentrations of compound 3.

FIG. 4 shows the effect of compound 3 on STAT5 phosphorylation in HEL cells. HEL cells were incubated with vehicle only, increasing concentrations of compound 3 or a positive control compound. The Western blots were treated with a STAT5 phosphospecific antibody and exposed to film for 5 minutes (top blot) and 1 minute (middle blot). The bottom blot shows total STAT protein, regardless of phosphorylation state. These blots clearly show a decrease in STAT5 phosphorylation in HEL Cells with increased concentrations of compound 3.

FIG. 5 shows the effect of treatment with compound 3 on growth hormone-stimulated insulin-like growth factor-1 (IGF-1) concentrations in mouse plasma.

FIG. 6 shows the efficacy of orally administered compound 3 in a subcutaneous tumour model of Ba/F3 TelJAK2 cells in nude mice.

FIG. 7 shows dot plots that demonstrate STAT5 phosphorylation (y axis) plotted against the expression of CD71 (x axis) in erythroid cells from the bone marrow of a patient with JAK2 V617F positive ET, as well as the effect of compound 3 on pYSTAT5. In this case, the negative control (A) shows only a small amount of pYSTAT5 staining that increases significantly after stimulation with erythropoietin (B) (the positive control). Addition of compound 3 caused a dose-dependent increase in inhibition of pYSTAT5 as illustrated in (C′). This is presented as the percentage inhibition of the measured pYSTAT5 activity of the positive control in the left panel and as an absolute shift in fluorescence intensity in the whole erythroid population in the right panel.

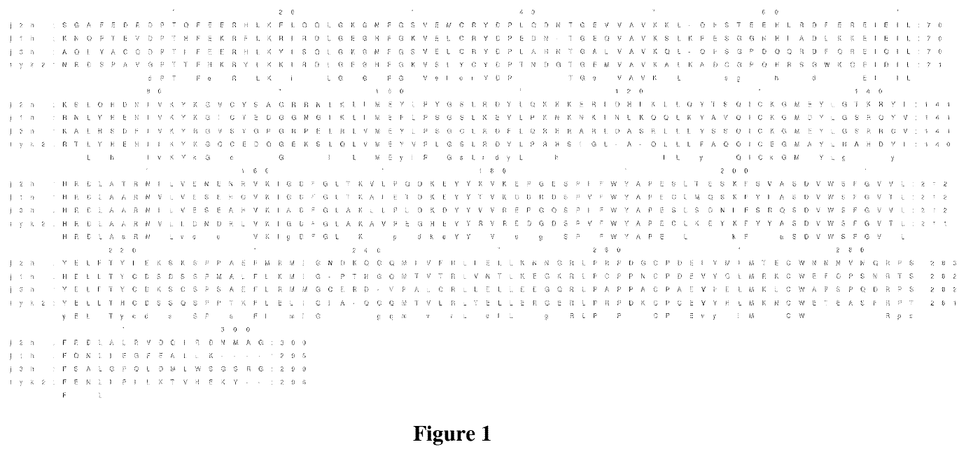

### MODES OF CARRYING OUT THE INVENTION

The present invention relates to compounds of formula I which inhibit kinases, in particular JAK kinases such as JAK2 and are useful in the treatment of kinase associated diseases such as immunological and inflammatory diseases including organ transplants; hyperproliferative diseases including cancer and myeloproliferative diseases; viral diseases; metabolic diseases; and vascular diseases.

### Compounds

The present invention relates to compounds of formula I

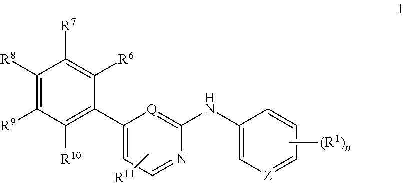

wherein
Q and Z are independently selected from N and CR¹;
n is 1, 2 or 3;
R¹ is independently selected from hydrogen, halogen, R², OR², OH, R⁴, OR⁴, CN, CF₃, (CH₂)ₙN(R²)₂, NO₂, R²R⁴, SO₂R⁴, NR²SO₂R³, COR⁴, NR²COR³, CO₂H, CO₂R², NR²COR⁴, R²CN, R²CN, R²OH, R²OR³ and OR⁵R⁴; or
two R¹ substituents together with the carbons which they are attached to form an unsaturated 5 or 6 membered heterocyclyl;
R² is substituted or unsubstituted C₁₋₄alkyl or substituted or unsubstituted C₁₋₄ alkylene where up to 2 carbon atoms can be optionally replaced with CO, NR^{Y}, CONR^{Y}, S, SO₂ or O;
R³ is R², C₂₋₄alkenyl or substituted or unsubstituted aryl;
R⁴ is NH₂, NHR², N(R¹)₂, substituted or unsubstituted morpholino, substituted or unsubstituted thiomorpholino, substituted or unsubstituted thiomorpholino-1-oxide, substituted or unsubstituted thiomorpholino-1,1-dioxide, substituted or unsubstituted piperazinyl, substituted or unsubstituted piperidinyl, substituted or unsubstituted pyridinyl, substituted or unsubstituted pyrrolidinyl, substituted or unsubstituted pyrrolyl, substituted or unsubstituted oxazolyl, substituted or unsubstituted imidazolyl, substituted or unsubstituted tetrahydrofuranyl and substituted or unsubstituted tetrahydropyranyl;
R⁵ is substituted or unsubstituted C₁₋₄alkylene;
R⁶-R¹⁰ are independently selected from H, R^{X}CN, halogen, substituted or unsubstituted C₁₋₄alkyl, OR¹, CO₂R¹, N(R¹)₂, NO₂, CON(R¹)₂, SO₂N(R^{Y})₂, N(SO₂R¹)₂, substituted or unsubstituted piperazinyl, N(R^{Y})SO₂R² and CF₃;
R^{x} is absent or substituted or unsubstituted C₁₋₆alkylene wherein up to 2 carbon atoms can be optionally replaced with CO, NSO₂R¹, NR^{Y}, CONR^{Y}, S, SO₂ or O;
R^{Y} is H or substituted or unsubstituted C₁₋₄alkyl; and
R¹¹ is selected from H, halogen, substituted or unsubstituted C₁₋₄ alkyl, OR², CO₂R², CN, CON(R¹)₂ and CF₃,
or an enantiomer thereof, a prodrug thereof or a pharmaceutically acceptable salt thereof.

In one embodiment, the compound of formula I has the formula Ia:

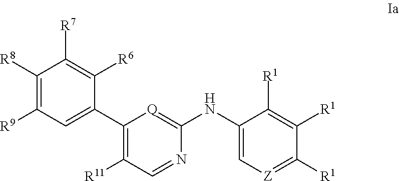

wherein,
Q and Z are independently selected from N and CR¹;
R¹ is independently selected from H, halogen, R², OR², OH, R⁴, CN, CF₃, NO₂, R²R⁴, SO₂R⁴, NR²SO₂R³, COR⁴, CO₂H, CO₂R², NR²COR³, NR²COR⁴, R²CN, R²OH, R²OR³ and OR⁵R³; or
two R¹ substitutents together with the carbon atoms to which they are attached form an unsaturated N-containing 5 or 6-membered heterocyclyl;
R² is C₁₋₄alkyl, or C₁₋₄alkylene;
R³ is R², C₂₋₄alkenyl or aryl;
R⁴ is NH₂, NHR², N(R²)², morpholino, thiomorpholino, thiomorpholino-1-oxide, thiomorpholino-1,1-dioxide, 4-carbonylmethyl piperazinyl, 4-methyl piperazinyl, 3- or 4-hydroxy piperidinyl, 4 hydroxymethyl piperidinyl, 4-pyrrolidinyl piperidinyl, 4 or 5-methyl oxazolyl, 4-hydroxy pyridinyl, 3-hydroxy pyrrolyl, 3-hydroxy pyrrolidinyl, pyridinyl pyrazolyl or imidazolyl;
R⁵ is C₂₋₄alkylene;
R⁶-R⁹ are independently selected from H, RCN, halogen, substituted or unsubstituted C₁₋₄alkyl, substituted or unsubstituted aryl, OR¹, CO₂R¹, N(R¹)₂, NO₂, CON(R¹)₂ and CON(R¹)₂;
R^{X} is substituted or unsubstituted C₁₋₄alkylene wherein up to 2 carbon atoms can be optionally replaced with CO, NSO₂R¹, NR^{Y}, CONR^{Y}, SO, SO₂, or O;
R^{Y} is H or substituted or unsubstituted C₁₋₄alkyl; and
R¹¹ is selected from H, halogen, substituted or unsubstituted C₁₋₄ alkyl, OR², CO₂R², CN, CON(R¹)₂ and CF₃,
or an enantiomer thereof, a prodrug thereof or a pharmaceutically acceptable salt thereof.

Preferably Q is N and Z is CR¹.

Preferably R¹ is hydrogen, morpholinyl, CH₂morpholinyl, C₁₋₄alkoxy, thiomorpholinyl, 3-hydroxypyrrolidinyl, iodo, fluoro, OH, 4-hydroxy piperidinyl, 4 hydroxymethyl piperidinyl, N-methyl piperidinyl, 3-hydroxy piperidinyl, carbonyl 4-pyrrolidinyl piperidinyl, oxy-4-piperidinyl, 4-carbonylmethyl piperazinyl, 4-methyl piperazinyl, 4-NHSO₂CH₃-piperidinyl, 4-oxy piperidinyl, imidazolyl CON(R¹)₂, CF₃ or R²OR³.

Preferably R⁶ is H or methyl.

Preferably R⁷ is H, methyl, methoxy, halogen such as chloro or hydroxy.

Preferably R⁸ is H, R^{X}CN such as CONHCN, CH₂NHCOCN, CN, CONHC(CH₃)₂CN, NCNSO₂CH₃, SO₂NHCH₂CN or N(SO₂CH₃)CH₂CN, OH, CO₂CH₂CH₃, CON(R¹)₂, N(R¹)₂ or CO₂R¹.

Preferably R⁹ is H, RCN such as CONHCN, CH₂NHCOCN or CH₂NHCN, methoxy halogen, OCF₃ or CF₃.

Preferably R¹¹ is H, halogen, substituted or unsubstituted C₁₋₄alkyl, OR², CO₂R², CN or CF₃, more preferably H, methyl, methoxy, Cl, Br, F or CO₂R², most preferably H or methyl.

In a preferred embodiment, the compound of formula I or Ia has the formula Ib:

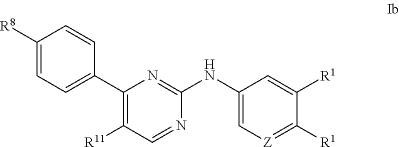

wherein
Z is independently selected from N and CH;
R¹ is independently selected from H, halogen, OH, CONHR², CON(R²)₂, CF₃, R²OR², CN, morpholino, thiomorpholinyl, thiomorpholino-1,1-dioxide, substituted or unsubstituted piperidinyl, substituted or unsubstituted piperazinyl, imidazolyl, substituted or unsubstituted pyrrolidinyl and C₁₋₄alkylene wherein the carbon atoms are optionally replaced with NR^{Y} and/or O substituted with morpholino, thiomorpholinyl, thiomorpholino-1,1-dioxide, substituted or unsubstituted piperidinyl, substituted or unsubstituted piperazinyl, imidazolyl or substituted or unsubstituted pyrrolidinyl;
R² is substituted or unsubstituted C₁₋₄alkyl;
R^{Y} is H or substituted or unsubstituted C₁₋₄alkyl;
R⁸ is RCN;
R^{X} is substituted or unsubstituted C₁₋₄alkylene wherein up to 2 carbon atoms can be optionally replaced with CO, NSO₂R¹, NR^{Y}, CONR^{Y}, SO, SO₂ or O;
R¹¹ is H or C₁₋₄alkyl,
or an enantiomer thereof, a prodrug thereof or a pharmaceutically acceptable salt thereof.

Examples of compounds of formula I include, but are not limited to, the following:

| Compound | | Exact | | LC- | |
|---|---|---|---|---|---|
| No. | Structure | mass | ¹H NMR | MS | Name |
| 1 | 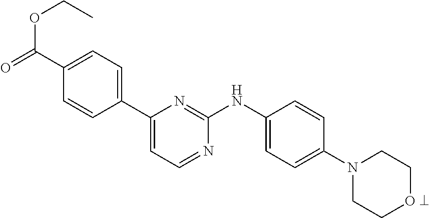 [SMILES: CCOC(=O)c1ccc(-c2ccnc(Nc3ccc(N4CCOCC4)cc3)n2)cc1] | 404.18 | ¹H NMR (300 MHz, d₆- DMSO): δ 9.49 (1H, s), 8.54 (1H, d, 5.0 Hz), 8.27 (2H, d, J = 8.7 Hz), 8.10 (2H, d, J = 8.7 Hz), 7.66 (2H, d, J = 9.1 Hz), 7.38 (1H, d, J = 5.0 Hz), 6.93 (2H, d, J = 8.7 Hz), 4.35 (2H, q, J = 6.9 Hz), 3.73 (4H, m), 3.04 (4H, m), 1.34 (3H, t, J = 6.9 Hz). | m/z 404.3 M⁺ | ethyl 4-(2-(4- morpholinophenyl- amino)pyrimidin- 4-yl)benzoate |
| 2 | 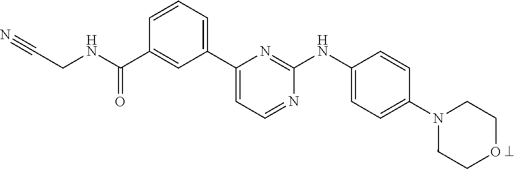 [SMILES: N#CCNC(=O)c1cccc(-c2ccnc(Nc3ccc(N4CCOCC4)cc3)n2)c1] | 414.18 | ¹H NMR (300 MHz, d₆- DMSO): δ 9.46 (1H, s), 9.34 (1H, s), 8.60 (1H, s), 8.53 (1H, d, J = 5.1 Hz), 8.32 (1H, d, J = 7.8 Hz), 7.99 (1H, d, J = 7.8 Hz), 7.67 (3H, m), 7.68 (1H, d, J = 5.1 Hz), 6.92 (2H, d, J = 9.0 Hz), 4.37 (2H, brs), 3.74 (4H, m), 3.04 (4H, m). | m/z 414.3 M⁺ | N-(cyanomethyl)- 3-(2-(4- morpholinophenyl- amino)pyrimidin- 4-yl)benzamide |
| 3 | 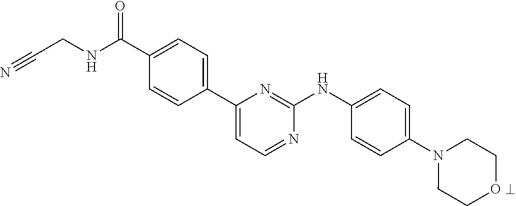 [SMILES: N#CCNC(=O)c1ccc(-c2ccnc(Nc3ccc(N4CCOCC4)cc3)n2)cc1] | 414.18 | ¹H NMR (300 MHz, d₆- DMSO): δ 9.47 (1H, s), 9.32 (1H, t, J = 5.5 Hz), 8.54 (1H, d, J = 5.0 Hz), 8.27 (2H, d, J = 8.7 Hz), 8.02 (2H, d, J = 8.2 Hz), 7.67 (2H, d, J = 9.1 Hz), 7.41 (1H, d, J = 5.5 Hz), 6.93 (2H, d, J = 9.1 Hz), 4.36 (2H, d, J = 5.5 Hz), 3.75 (4H, m), 3.05 (4H, m). | m/z 415.3 [M + H]⁺ | N-(cyanomethyl)- 4-(2-(4- morpholinophenyl- amino)pyrimidin- 4-yl)benzamide |
| 4 | 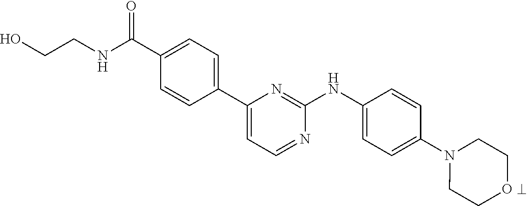 [SMILES: O=C(NCCO)c1ccc(-c2ccnc(Nc3ccc(N4CCOCC4)cc3)n2)cc1] | 419.20 | ¹H NMR (300 MHz, d₆- DMSO): δ 8.42 (1H, d, J = 5.2 Hz), 8.21 (2H, d, J = 8.4 Hz), 7.95 (2H, d, J = 8.4 Hz), 7.60 (2H, d, J = 9.0 Hz), 7.27 (1H, d, J = 5.2 Hz), 7.27 (1H, d, J = 5.2 Hz), 6.98 (2H, d, J = 9.0 Hz), 3.84 (4H, m), 3.73 (2H, t, J = 5.8 Hz), 3.53 (2H, t, J = 5.8 Hz), 3.11 (4H, m). | m/z 419.4 M⁺ | N-(2- hydroxyethyl)-4- (2-(4- morpholinophenyl- amino)pyrimidin- 4-yl)benzamide |
| 5 | 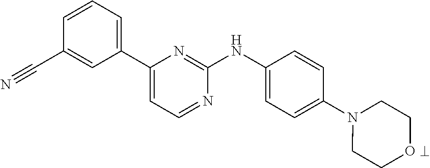 [SMILES: N#Cc1cccc(-c2ccnc(Nc3ccc(N4CCOCC4)cc3)n2)c1] | 357.16 | ¹H NMR (300 MHz, CDCl₃): δ 8.49 (d, J = 5.1 Hz, 1H), 8.37- 8.36 (m, 1H), 8.28-8.25 (m, 1H), 7.78-7.75 (m, 1H), 7.63- 7.61 (m, 1H), 7.57-7.54 (m, 2H), 7.09 (d, J = 4.8 Hz, 1H), 7.00-6.97 (m, 2H), 3.89 (t, J = 4.5 Hz, 4H), 3.16 (t, J = 4.9 Hz, 4H). | m/z 356.8 M⁺ | 3-(2-(4- morpholinophenyl- amino)pyrimidin- 4-yl)benzonitrile |
| 6 | 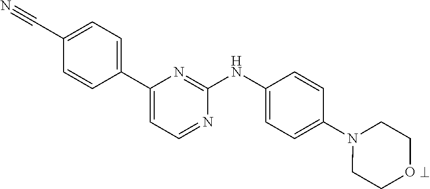 [SMILES: N#Cc1ccc(-c2ccnc(Nc3ccc(N4CCOCC4)cc3)n2)cc1] | 357.16 | ¹H NMR (300 MHz, CDCl₃): δ 8.50 (d, J = 5.1 Hz, 1H), 8.15 (d, J = 8.7 Hz, 2H), 7.78 (d, J = 8.7 Hz, 2H), 7.58-7.55 (m, 2H), 7.13-7.11 (m, 1H), 7.01-6.98 (m, 2H), 3.90 (t, J = 4.5 Hz, 4H), 3.16 (t, J = 4.2 Hz, 4H). | m/z 356.8 M⁺ | 4-(2-(4- morpholinophenyl- amino)pyrimidin- 4-yl)benzonitrile |
| 7 | 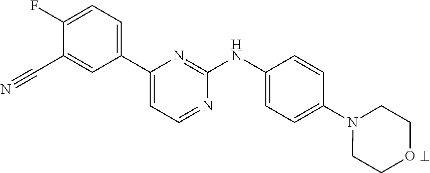 [SMILES: N#Cc1cc(-c2ccnc(Nc3ccc(N4CCOCC4)cc3)n2)ccc1F] | 375.15 | ¹H NMR (300 MHz, CDCl₃): δ 8.49 (d, J = 5.7 Hz, 1H), 8.36 (dd, J = 5.7, 2.4 Hz, 1H), 8.29 (m, 1H), 7.54 (d, J = 9.3 Hz, 1H), 7.33 (t, J = 9.0 Hz, 1H), 7.10 (br. s, 1H), 7.05 (d, J = 5.1 Hz, 1H), 6.97 (d, J = 8.7 Hz, 2H), 3.88 (t, J = 5.1 Hz, 4H), 3.15 (t, J = 5.4 Hz, 4H). | m/z 375.0 M⁺ | 2-fluoro-5-(2-(4- morpholinophenyl- amino)pyrimidin- 4-yl)benzonitrile |
| 8 | 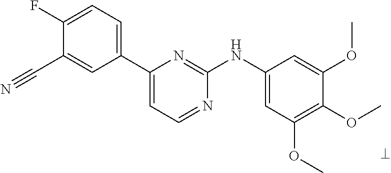 [SMILES: COc1cc(Nc2nccc(-c3ccc(F)c(C#N)c3)n2)cc(OC)c1OC] | 380.13 | ¹H NMR (300 MHz, CDCl₃): δ 8.53 (d, J = 5.4 Hz, 1H), 8.44 (dd, J = 5.7 Hz, 2.1 Hz, 1H), 8.29 (m, 1H), 7.34 (t, J = 8.7 Hz, 1H), 7.19 (br. s, 1H), 7.12 (d, J = 5.1 Hz, 1H), 7.00 (s, 2H), 3.92 (s, 6H), 3.85 (s, 3H). | m/z 379.9 M⁺ | 2-fluoro-5-(2- (3,4,5- trimethoxyphenyl- amino)pyrimidin-4- yl)benzonitrile |
| 9 | 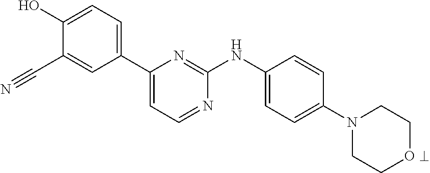 [SMILES: N#Cc1cc(-c2ccnc(Nc3ccc(N4CCOCC4)cc3)n2)ccc1O] | 373.15 | ¹H NMR (300 MHz, CDCl₃): δ 8.39 (m, 1H), 8.23 (d, J = 1.8 Hz, 1H), 8.11 (dd, J = 8.7, 2.1 Hz, 1H), 7.57-7.55 (m, 2H), 7.05-7.02 (m, 2H), 7.01-6.90 (m, 2H), 3.89 (t, J = 4.5 Hz, 4H), 3.41 (m, 1H), 3.15-3.13 (m, 4H). | m/z 373.0 M⁺ | 2-hydroxy-5-(2- (4- morpholinophenyl- amino)pyrimidin- 4-yl)benzonitrile |
| 10 | 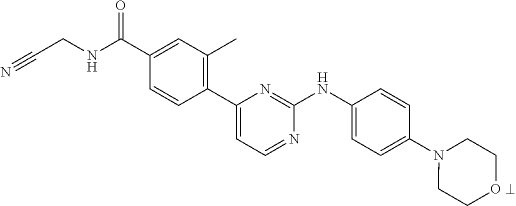 [SMILES: Cc1cc(C(=O)NCC#N)ccc1-c1ccnc(Nc2ccc(N3CCOCC3)cc2)n1] | 428.20 | ¹H NMR (300 MHz, CDCl₃): δ 8.45 (1H, d, J = 5.0 Hz), 7.72 (1H, d, J = 1.6 Hz), 7.76 (1H, dd, J = 1.6, 8.0 Hz), 7.52 (3H, m), 7.14 (1H, s), 6.91 (2H, d, J = 9.0 Hz), 6.77 (1H, d, J = 5.0 Hz), 6.67 (1H, t, J = 5.7 Hz), 4.39 (2H, d, J = 5.7 Hz), 3.86 (4H, m), 3.11 (4H, m), 2.48 (3H, s). | m/z 428.3 M⁺ | N-(cyanomethyl)- 3-methyl-4-(2-(4- morpholinophenyl- amino)pyrimidin- 4-yl)benzamide |
| 11 | 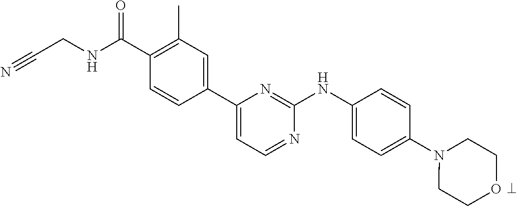 [SMILES: Cc1cc(-c2ccnc(Nc3ccc(N4CCOCC4)cc3)n2)ccc1C(=O)NCC#N] | 428.20 | ¹H NMR (300 MHz, 1:1 CDCl₃ d₄-MeOH): δ 8.42 (1H, d, J = 5.2 Hz), 7.99 (1H, brs), 7.96 (1H, dd, J = 1.2, 8.1 Hz), 7.62 (2H, d, J = 9.2 Hz),* 7.53 (1H, d, J = 8.0 Hz), 7.19 (1H, d, J = 5.2 Hz), 6.99 (2H, d, J = 9.2 Hz), 4.33 (2H, s), 3.89 (4H, m), 3.15 (4H, m), 2.54 (3H, s). * Partially obscured by CHCl₃ signal. | m/z 428.3 M⁺ | N-(cyanomethyl)- 2-methyl-4-(2-(4- morpholinophenyl- amino)pyrimidin- 4-yl)benzamide |
| 12 | 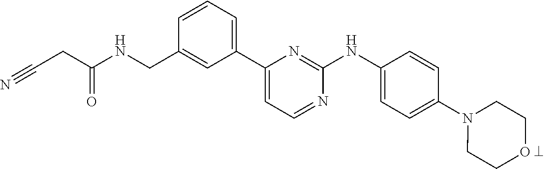 [SMILES: N#CCC(=O)NCc1cccc(-c2ccnc(Nc3ccc(N4CCOCC4)cc3)n2)c1] | 428.20 | ¹H NMR (300 MHz, d₆- DMSO): δ 9.40 (1H, s), 8.78 (1H, dd, J = 5.5, 5.9 Hz), 8.48 (1H, d, J = 5.5 Hz), 8.03 (2H, m), 7.67 (2H, d, J = 9.1 Hz), 7.50 (1H, t, J = 7.8 Hz), 7.43 (1H, m), 7.29 (1H, d, J = 5.0 Hz), 6.93 (2H, d, J = 9.1 Hz), 4.39 (2H, d, J = 5.9 Hz), 3.73 (4H, m), 3.71 (2H, s), 3.04 (4H, m). | m/z 428.2 M⁺ | 2-cyano-N-(3-(2- (4- morpholinophenyl- amino)pyrimidin- 4- yl)benzyl)acetamide |
| 13 | 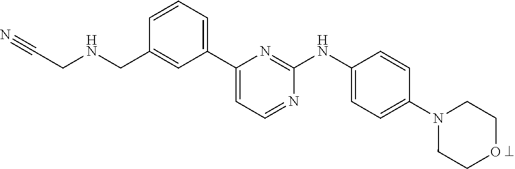 [SMILES: N#CCNCc1cccc(-c2ccnc(Nc3ccc(N4CCOCC4)cc3)n2)c1] | 400.20 | ¹H NMR (300 MHz, d₆- DMSO): δ 9.41 (1H, s), 8.47 (1H, d, J = 5.0 Hz), 8.13 (1H, brs), 8.02 (1H, ddd, J = 1.8, 4.1, 5.0 Hz), 7.68 (2H, d, J = 9.1 Hz), 7.49 (2H, brd, J = 4.5 Hz), 7.31 (1H, d, J = 5.0 Hz), 6.92 (2H, d, J = 9.1 Hz), 3.85 (2H, d, J = 5.9 Hz), 3.73 (4H, m), 3.63 (2H, d, J = 7.3 Hz), 3.03 (4H, m). | m/z 400.1 M⁺ | 2-(3-(2-(4- morpholinophenyl- amino)pyrimidin- 4- yl)benzylamino) acetonitrile |
| 14 | 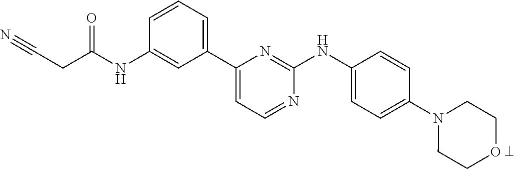 [SMILES: N#CCC(=O)Nc1cccc(-c2ccnc(Nc3ccc(N4CCOCC4)cc3)n2)c1] | 414.18 | ¹H NMR (300 MHz, d₆- DMSO): δ 10.46 (1H, s), 9.41 (1H, s), 8.53 (1H, s), 8.49 (1H, d, J = 5.5 Hz), 7.83 (1H, d, J = 7.8 Hz), 7.72 (2H, d, J = 9.1 Hz), 7.58 (1H, brd, J = 8.2 Hz), 7.48 (1H, dd, J = 7.8, 7.8 Hz), 7.24 (1H, d, J = 5.0 Hz), 6.96 (2H, d, J = 9.1 Hz), 3.95 (2H, s), 3.73 (4H, m), 3.04 (4H, m). | m/z 414.3 M⁺ | 2-cyano-N-(3-(2- (4- morpholinophenyl- amino)pyrimidin- 4- yl)phenyl)acetamide |
| 15 | 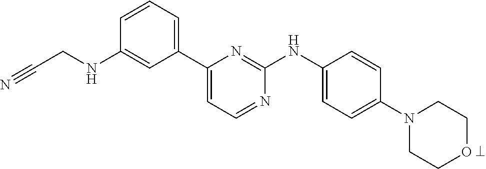 [SMILES: N#CCNc1cccc(-c2ccnc(Nc3ccc(N4CCOCC4)cc3)n2)c1] | 386.19 | ¹H NMR (300 MHz, d₆- acetonitrile): δ 8.42 (1H, d, J = 5.0 Hz), 7.72 (1H, br), 7.64 (2H, d, J = 9.1 Hz), 7.51-7.54 (2H, m), 7.37 (1H, dd, J = 7.8, 8.2 Hz), 7.20 (1H, d, J = 5.0 Hz), 6.98 (2H, m), 6.90 (1H, m), 5.04 (1H, t, J = 6.9 Hz), 4.22 (2H, d, J = 6.9 Hz), 3.79 (4H, m), 3.08 (4H, m). | m/z 386.2 M⁺ | 2-(3-(2-(4- morpholinophenyl- amino)pyrimidin- 4- yl)phenylamino) acetonitrile |
| 16 | 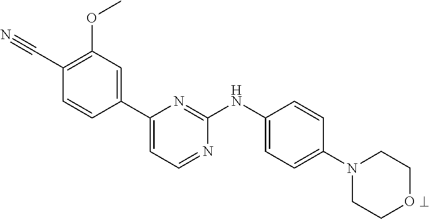 [SMILES: COc1cc(-c2ccnc(Nc3ccc(N4CCOCC4)cc3)n2)ccc1C#N] | 387.17 | ¹H NMR (300 MHz, CDCl₃): δ 8.50 (d, J = 5.1 Hz, 1H), 7.79 (d, J = 1.2 Hz, 1H), 7.67 (d, J = 8.0 Hz, 1H), 7.61 (dd, J = 1.4, 8.0 Hz, 1H), 7.56 (d, J = 9.0 Hz, 2H), 7.12 (br. s, 1H), 7.10 (d, J = 5.4 Hz, 1H), 6.95 (d, J = 9.0 Hz, 2H), 4.05 (s, 3H), 3.89 (m, 4H), 3.14 (m, 4H). | m/z 388.2 [M + H]⁺ | 2-methoxy-4-(2- (4- morpholinophenyl- amino)pyrimidin- 4-yl)benzonitrile |
| 17 | 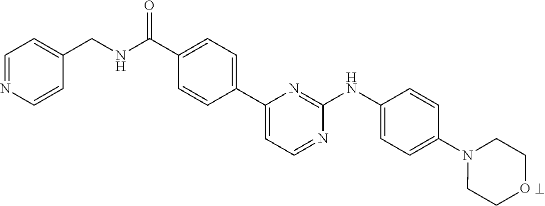 [SMILES: O=C(NCc1ccncc1)c1ccc(-c2ccnc(Nc3ccc(N4CCOCC4)cc3)n2)cc1] | 466.21 | ¹H NMR (300 MHz, d₆- DMSO): δ 9.47 (1H, s), 9.24 (1H, t, J = 5.9 Hz), 8.52 (1H, d, J = 5.5 Hz), 8.51 (2H, m), 8.24 (2H, d, J = 8.2 Hz), 8.05 (2H, d, J = 8.7 Hz), 7.66 (2H, d, J = 9.1 Hz), 7.39 (1H, d, J = 5.5 Hz), 7.32 (2H, d, J = 5.9 Hz), 6.92 (2H, d, J = 9.1 Hz), 4.52 (2H, d, J = 5.9 Hz), 3.74 (4H, m), 3.04 (4H, m). | m/z 467.1 [M + H]⁺ | 4-(2-(4- morpholinophenyl- amino)pyrimidin- 4-yl)-N-(pyridin- 4- ylmethyl)benzamide |
| 18 | 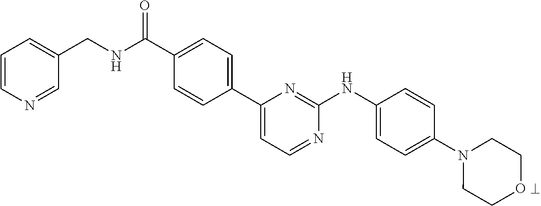 [SMILES: O=C(NCc1cccnc1)c1ccc(-c2ccnc(Nc3ccc(N4CCOCC4)cc3)n2)cc1] | 466.21 | ¹H NMR (300 MHz, d₆- DMSO): δ 9.45 (1H, s), 9.20 (1H, t, J = 5.9 Hz), 8.57 (1H, d, J = 1.8 Hz), 8.52 (1H, d, J = 5.0 Hz), 8.46 (1H, dd, J = 1.8, 5.0 Hz), 8.23 (2H, d, J = 8.7 Hz), 8.03 (2H, d, J = 8.7 Hz), 7.74 (1H, ddd, J = 1.8, 2.8, 7.8 Hz), 7.66 (2H, d, J = 9.1 Hz), 7.38 (1H, d, J = 5.0 Hz), 7.36 (1H, m), 6.92 (2H, d, J = 9.1 Hz), 4.52 (2H, d, J = 5.9 Hz), 3.73 (4H, m), 3.04 (4H, m). | m/z 467.1 [M + H]⁺ | 4-(2-(4- morpholinophenyl- amino)pyrimidin- 4-yl)-N-(pyridin- 3- ylmethyl)benzamide |
| 19 | 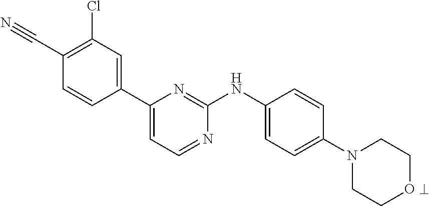 [SMILES: N#Cc1ccc(-c2ccnc(Nc3ccc(N4CCOCC4)cc3)n2)cc1Cl] | 391.12 | ¹H NMR (300 MHz, CDCl₃): δ 8.51 (d, J = 5.1 Hz, 1H), 8.21 (s, 1H), 8.02 (d, J = 9.6 Hz, 1H), 7.78 (d, J = 8.4 Hz, 1H), 7.53 (d, J = 9.0 Hz, 2H), 7.16 (br. s, 1H), 7.09 (d, J = 5.1 Hz, 1H), 6.96 (d, J = 9.0 Hz, 2H), 3.88 (m, 4H), 3.15 (m, 4H). | m/z 391.3/ 393.3 M⁺ | 2-chloro-4-(2-(4- morpholinophenyl- amino)pyrimidin- 4-yl)benzonitrile |
| 20 | 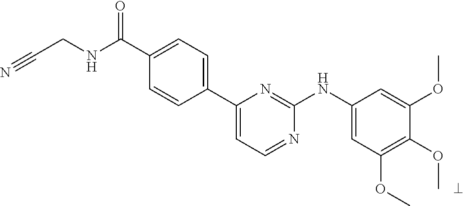 [SMILES: COc1cc(Nc2nccc(-c3ccc(C(=O)NCC#N)cc3)n2)cc(OC)c1OC] | 419.16 | ¹H NMR (300 MHz, d₆- DMSO): δ 9.59 (1H, s), 9.32 (1H, t, J = 5.5 Hz), 8.59 (1H, d, J = 5.0 Hz), 8.31 (2H, d, J = 8.7 Hz), 8.02 (2H, d, J = 8.7 Hz), 7.47 (1H, d, J = 5.0 Hz), 7.30 (2H, s), 4.34 (2H, d, J = 5.5 Hz), 3.80 (6H, s) 2 x OMe, 3.63 (2H, s) OMe. | m/z 420.3 [M + H]⁺ | N-(cyanomethyl)- 4-(2-(3,4,5- trimethoxyphenyl- amino)pyrimidin-4- yl)benzamide |
| 21 | 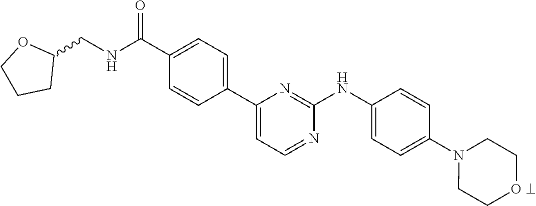 [SMILES: O=C(NCC1CCCO1)c1ccc(-c2ccnc(Nc3ccc(N4CCOCC4)cc3)n2)cc1] | 459.23 | ¹H NMR (300 MHz, d₆- DMSO): δ 9.45 (1H, s), 8.63 (1H, t, J = 5.9 Hz), 8.51 (1H, d, J = 5.0 Hz), 8.21 (2H, d, J = 8.7 Hz), 7.99 (2H, d, J = 8.2 Hz), 7.65 (2H, d, J = 9.1 Hz), 7.37 (1H, d, J = 5.0 Hz), 6.92 (2H, d, J = 9.1 Hz), 3.99 (1H, m), 3.79 (1H, m), 3.73 (4H, m), 3.62 (1H, m), 3.30 (2H, m),* 3.04 (4H, m), 1.97-1.76 (3H, m), 1.65-1.54 (1H, m). * Partially overlapping with water signal from solvent. | m/z 460.4 [M + H]⁺ | 4-(2-(4- morpholinophenyl- amino)pyrimidin- 4-yl)-N- ((tetrahydrofuran- 2- yl)methyl)benzamide |
| 22 | 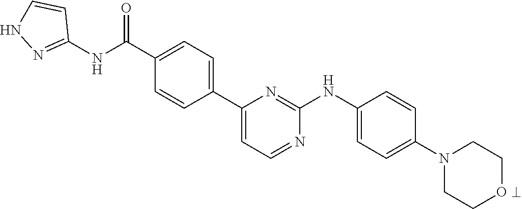 [SMILES: O=C(Nc1cc[nH]n1)c1ccc(-c2ccnc(Nc3ccc(N4CCOCC4)cc3)n2)cc1] | 441.19 | ¹H NMR (300 MHz, d₆- DMSO): δ 12.44 (1H, brs), 10.90 (1H, brs), 9.46 (1H, s), 8.52 (1H, d, J = 5.0 Hz), 8.24 (2H, d, J = 8.2 Hz), 8.14 (2H, d, J = 8.7 Hz), 7.47 (3H, brd, J = 9.1 Hz),* 7.40 (1H, d, J = 5.0 Hz), 6.93 (2H, d, J = 9.1 Hz), 6.66 (1H, brs), 3.74 (4H, m), 3.04 (4H, m). * Overlapping resonances 2H d and 1H m. | m/z 442.3 [M + H]⁺ | 4-(2-(4- morpholinophenyl- amino)pyrimidin- 4-yl)-N-(1H- pyrazol-3- yl)benzamide |
| 23 | 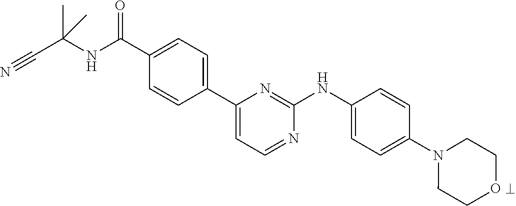 [SMILES: CC(C)(C#N)NC(=O)c1ccc(-c2ccnc(Nc3ccc(N4CCOCC4)cc3)n2)cc1] | 442.21 | ¹H NMR (300 MHz, d₆- DMSO): δ 9.47 (1H, s), 8.83 (1H, s), 8.53 1H, d, J = 5.0 Hz), 8.25 (2H, d, J = 8.7 Hz), 8.01 (2H, d, J = 8.2 Hz), 7.66 (2H, d, J = 9.1 Hz), 7.39 (1H, d, J = 5.0 Hz), 6.92 (2H, d, J = 9.1 Hz), 3.73 (4H, m), 3.04 (4H, m), 1.71 (6H, s). | m/z 443.4 [M + H]⁺ | N-(2- cyanopropan-2- yl)-4-(2-(4- morpholinophenyl- amino)pyrimidin- 4-yl)benzamide |
| 24 | 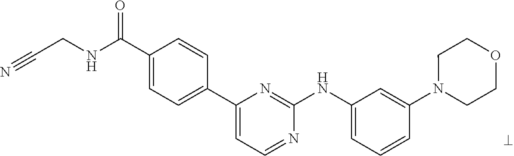 [SMILES: N#CCNC(=O)c1ccc(-c2ccnc(Nc3cccc(N4CCOCC4)c3)n2)cc1] | 414.18 | ¹HNMR (500 MHz. d₆-DMSO): δ 9.58 (s, 1H), 9.33 (t, J = 5.5 Hz, 1H), 8.59 (d, J = 5.0 Hz, 1H), 8.29 (d, J = 8.3 Hz, 2H), 8.02 (d, J = 5.1 Hz, 2H), 7.63 (s, 1H), 7.47 (d, J = 5.5 Hz, 1H), 7.25 (m, 1H), 7.16 (t, J = 8.0 Hz, 1H), 6.59 (dd, J = 8.0, 2.0 Hz, 1H), 4.36 (d, J = 5.5 Hz, 2H), 3.77 (m, 4H), 3.13 (m, 4H). | m/z 415.4 [M + H]⁺ | N-(cyanomethyl)- 4-(2-(3- morpholinophenyl- amino)pyrimidin- 4-yl)benzamide |
| 25 | 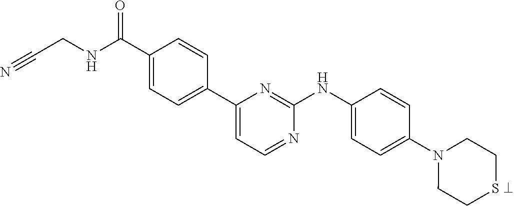 [SMILES: N#CCNC(=O)c1ccc(-c2ccnc(Nc3ccc(N4CCSCC4)cc3)n2)cc1] | 430.16 | ¹H NMR (300 MHz, d₆- DMSO): δ 9.48 (s, 1H), 9.32 (t, J = 5.4 Hz, 1H), 8.53 (d, J = 4.8 Hz, 1H), 8.26 (d, J = 8.7 Hz, 2H), 8.02 (d, J = 8.7 Hz, 2H), 7.66 (d, J = 9.3 Hz, 2H), 7.40 (d, J = 5.1 Hz, 1H), 6.92 (d, J = 9.0 Hz, 211), 4.35 (d, J = 5.7 Hz, 2H), 3.41 (m, 4H), 2.70 (m, 4H). | m/z 431.3 [M + H]⁺ | N-(cyanomethyl)- 4-(2-(4- thiomorpholino- phenylamino) pyrimidin-4- yl)benzamide |
| 26 | 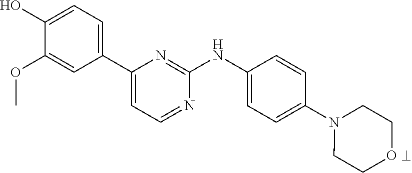 [SMILES: COc1cc(-c2ccnc(Nc3ccc(N4CCOCC4)cc3)n2)ccc1O] | 378.17 | ¹H NMR (300 MHz, CDCl₃): δ 8.37 (1H, d, J = 5.4 Hz), 7.74 (1H, d, J = 1.5 Hz), 7.54-7.60 (3H, m), 6.98-7.07 (3H, m), 6.93 (2H, d, J = 8.7 Hz), 5.89 (1H, bs), 4.00 (3H, s), 3.88 (4H, m), 3.13 (4H, m). | m/z 378.4 M⁺ | 2-methoxy-4-(2- (4- morpholinophenyl- amino)pyrimidin- 4-yl)phenol |
| 27 | 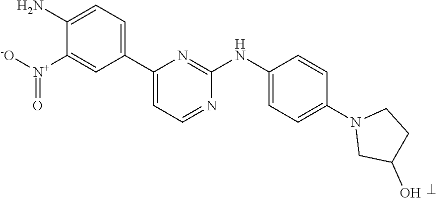 | 392.16 | | LC- ESI- MS (method B): rt 6.4 min, m/z 393.1 [M + H]⁺ | 1-(4-(4-(4-amino- 3- nitrophenyl) pyrimidin-2- ylamino)phenyl) pyrrolidin-3-ol |
| 28 | 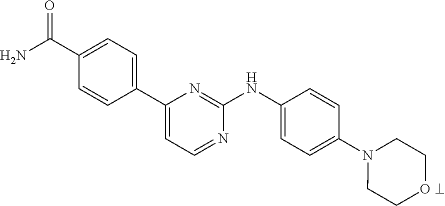 [SMILES: NC(=O)c1ccc(-c2ccnc(Nc3ccc(N4CCOCC4)cc3)n2)cc1] | 375.17 | ¹H NMR (300 MHz, d₆- DMSO): δ 9.46 (s, 1H), 8.51 (d, J = 5.2 Hz, 1H), 8.20 (d, J = 8.4 Hz, 2H), 8.07 (brs, 1H), 8.01 (d, J = 8.4 Hz, 2H), 7.66 (d, J = 9.0 Hz, 2H), 7.47 (brs, 1H), 7.39 (d, J = 4.8 Hz, 1H), 6.92 (d, J = 9.1 Hz, 2H), 3.73 (m, 4H), 3.04 (m, 4H). | m/z 376.1 [M + H]⁺ and m/z 374.2 [M − H]⁻ | 4-(2-(4- morpholinophenyl- amino)pyrimidin- 4-yl)benzamide |
| 29 | 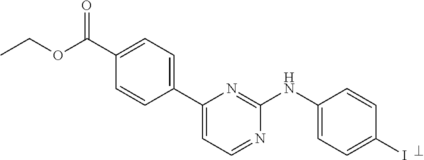 [SMILES: CCOC(=O)c1ccc(-c2ccnc(Nc3ccc(I)cc3)n2)cc1] | 445.03 | ¹H NMR (500 MHz, d₆- DMSO): δ 9.91 (s, 1H), 8.63 (d, J = 5.0 Hz, 1H), 8.29 (d, J = 8.0 Hz, 2H), 8.11 (d, J = 8.5 Hz, 2H), 7.70 (d, J = 9.0 Hz, 2H), 7.64 (d, J = 9.0 Hz, 2H), 7.51 (d, J = 4.5 Hz, 1H), 4.36 (q, J = 7.0 Hz, 2H), 1.35 (t, J = 7.5 Hz, 3H). | m/z found 446.2 [M + H]⁺ | ethyl 4-(2-(4- iodophenylamino) pyrimidin-4- yl)benzoate |
| 30 | 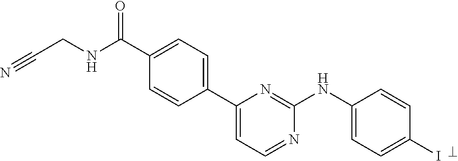 [SMILES: N#CCNC(=O)c1ccc(-c2ccnc(Nc3ccc(I)cc3)n2)cc1] | 455.02 | ¹H NMR (300 MHz, d₆- DMSO): δ 9.88 (s, 1H), 9.61 (t, J = 5.4 Hz, 1H), 8.61 (d, J = 5.1 Hz, 1H), 8.27 (d, J = 8.4 Hz, 2H), 8.09 (d, J = 8.4 Hz, 2H), 7.7 (d, J = 9.0 Hz, 2H), 7.63 (d, J = 9.0 Hz, 2H), 7.52 (d, J = 5.1 Hz, 1H), 4.33 (d, J = 5.4 Hz, 2H). | m/z 456.2 [M + H]⁺ | N-(cyanomethyl)- 4-(2-(4- iodophenylamino) pyrimidin-4- yl)benzamide |
| 31 | 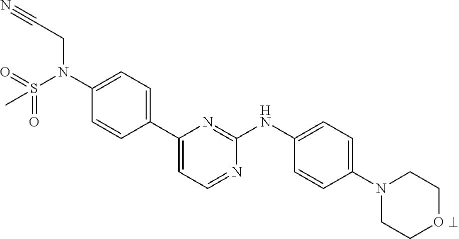 [SMILES: CS(=O)(=O)N(CC#N)c1ccc(-c2ccnc(Nc3ccc(N4CCOCC4)cc3)n2)cc1] | 464.16 | ¹H NMR (300 MHz, CDCl₃): δ 8.46 (d, J = 5.1 Hz, 1H), 8.15 (d, J = 8.7 Hz, 2H), 7.65 (d, J = 8.7 Hz, 2H), 7.56 (d, J = 9.3 Hz, 2H), 7.16 (bs, 1H), 7.09 (d, J = 5.4 Hz, 1H), 6.96 (d, J = 9.3 Hz, 2H), 4.00 (s, 2H), 3.90-3.87 (m, 4 H), 3.17-3.13 (m, 4H), 3.09 (s, 3H). | m/z 465.4 [M + H]⁺ | N-(cyanomethyl)- N-(4-(2-(4- morpholinophenyl- amino)pyrimidin- 4- yl)phenyl)methane- sulfonamide |
| 32 | 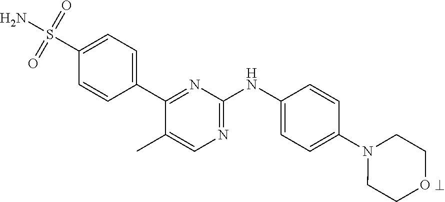 [SMILES: Cc1cnc(Nc2ccc(N3CCOCC3)cc2)nc1-c1ccc(S(N)(=O)=O)cc1] | 425.15 | ¹H NMR (300 MHz, d₆- DMSO): δ 9.36 (s, 1H), 8.39 (s, 1H), 7.95 (d, J = 8.1 Hz, 2H), 7.84 (d, J = 8.1 Hz, 2H), 7.61 (m, 2H), 7.48 (s, 2H), 6.88 (m, 2H), 3.72 (m, 4H), 3.01 (m, 4H), 2.19 (s, 3H). | m/z 426.3 [M + H]⁺ | 4-(5-methyl-2-(4- morpholinophenyl- amino)pyrimidin- 4- yl)benzene- sulfonamide |
| 33 | 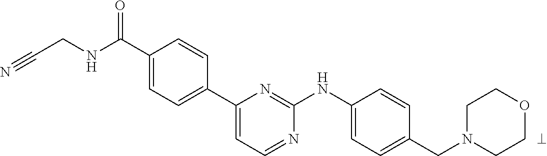 [SMILES: N#CCNC(=O)c1ccc(-c2ccnc(Nc3ccc(CN4CCOCC4)cc3)n2)cc1] | 428.20 | ¹H NMR (300 MHz, d₆- DMSO): δ 9.70 (s, 1H), 9.37- 9.31 (m, 1H), 8.59 (d, J = 5.1 Hz, 1H), 8.29 (d, J = 8.7 Hz, 2H), 8.03 (d, J = 9.0 Hz, 2H), 7.78 (d, J = 9.0 Hz, 2H), 7.47 (d, J = 5.1 Hz, 1H), 7.24 (d, J = 9.0 Hz, 2H), 4.35 (d, J = 5.7 Hz, 2H), 3.64-3.50 (m, 4H), 3.41 (s, 2H), 2.35 (brs, 4H). | m/z 429.3 [M + H]⁺ | N-(cyanomethyl 4-(2-(4- (morpholinomethyl) phenylamino) pyrimidin-4- yl)benzamide |
| 34 | 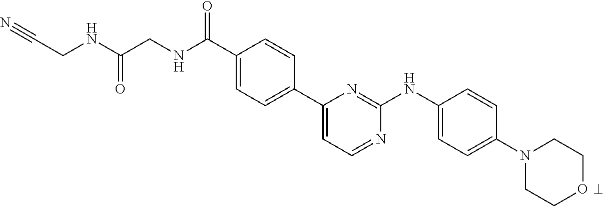 [SMILES: N#CCNC(=O)CNC(=O)c1ccc(-c2ccnc(Nc3ccc(N4CCOCC4)cc3)n2)cc1] | 471.20 | ¹H NMR (300 MHz, d₆- DMSO): δ 9.49 (s, 1H), 9.00 (t, J = 6.0, 1H), 8.67 (t, J = 5.4 Hz, 1H), 8.53 (d, J = 5.1 Hz, 1H), 8.26 (d, J = 8.7 Hz, 2H), 8.05 (d, J = 9.0 Hz, 2H), 7.67 (d, J = 9.0 Hz, 2H), 7.41 (d, J = 5.4 Hz, 1H), 6.94 (d, J = 9.0 Hz, 2H), 4.16 (d, J = 5.4 Hz, 2H), 3.95 (d, J = 5.7 Hz, 2H), 3.78- 3.72 (m. 4H), 3.08-3.02 (m, 4H). | m/z 472.4 [M + H]⁺ | N-(2- (cyanomethylamino)- 2-oxoethyl)-4- (2-(4- morpholinophenyl- amino)pyrimidin- 4-yl)benzamide |
| 35 | 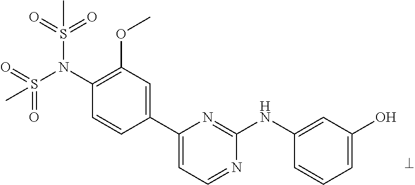 [SMILES: COc1cc(-c2ccnc(Nc3cccc(O)c3)n2)ccc1N(S(C)(=O)=O)S(C)(=O)=O] | 464.08 | ¹H NMR (300 MHz, CDCl₃/CD₃OD) δ 8.46 (d, J = 5.1 Hz, 1H), 7.92 (d, J = 1.5 Hz, 1H), 7.62 (dd, J = 1.8, J = 8.1 Hz, 1H), 7.41 (d, J = 8.1 Hz, 1H), 7.40 (m, 1H), 7.15 (m, 2H), 7.15 (d, J = 5.4 Hz, 1H), 6.55 (m, 1H), 4.05 (s, 3H), 3.47 (s, 6H). | m/z 465.2 [M + H]⁺ | N-(4-(2-(3- hydroxyphenyl- amino)pyrimidin-4- yl)-2- methoxyphenyl)- N- (methylsulfonyl) methanesulfonamide |
| 36 | 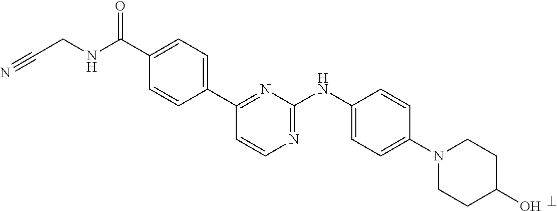 [SMILES: N#CCNC(=O)c1ccc(-c2ccnc(Nc3ccc(N4CCC(O)CC4)cc3)n2)cc1] | 428.20 | ¹H NMR (300 MHz, d₆- DMSO): δ 9.44 (s, 1H), 9.38- 9.30 (m, 1H), 8.52 (d, J = 5.1, 1H), 8.26 (d, J = 8.4 Hz, 2H), 8.02 (d, J = 8.7 Hz, 2H), 7.61 (d, J = 9.3 Hz, 2H), 7.38 (d, J = 5.1 Hz, 1H), 6.91 (d, J = 9.3 Hz, 2H), 4.69 (d, J = 4.2 Hz, 1H), 4.35 (d, J = 5.4 Hz, 2H), 3.68-3.50 (m, 2H), 2.82-2.68 (m, 2H), 1.93-1.74 (m, 2H), 1.58-1.39 (m, 2H). | m/z 429.3 [M + H]⁺ | N-(cyanomethyl)- 4-(2-(4-(4- hydroxypiperidin- 1- yl)phenylamino) pyrimidin-4- yl)benzamide |
| 37 | 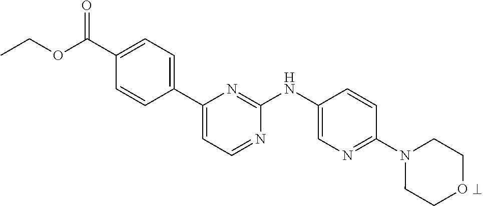 [SMILES: CCOC(=O)c1ccc(-c2ccnc(Nc3ccc(N4CCOCC4)nc3)n2)cc1] | 405.18 | ¹H NMR (300 MHz, d₆- DMSO): δ 9.54 (s, 1H), 8.54 (d, J = 5.7 Hz, 1H), 8.51 (d, J = 2.7 Hz, 1H), 8.25 (d, J = 8.1 Hz, 2H), 8.10 (d, J = 7.8 Hz, 2H), 7.98 (dd, J = 9.0 Hz, 2.7, 1H), 7.40 (d, J = 5.4 Hz, 1H), 6.87 (d, J = 9.0 Hz, 1H), 4.35 (q, J = 6.9 Hz, 2H), 3.74-3.68 (m, 4H), 3.40-3.33 (m, 4H), 1.34 (t, J = 7.5 Hz, 3H). | m/z 406.3 [M + H]⁺ | ethyl 4-(2-(6- morpholinopyridin- 3- ylamino)pyrimidin- 4-yl)benzoate |
| 38 | 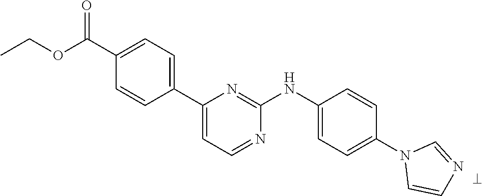 [SMILES: CCOC(=O)c1ccc(-c2ccnc(Nc3ccc(-n4ccnc4)cc3)n2)cc1] | 385.15 | ¹H NMR (300 MHz, d₆- DMSO): δ 9.98 (s, 1H), 8.65 (d, J = 4.8 Hz, 1H), 8.32 (d, J = 8.1 Hz, 2H), 8.17 (s, 1H), 8.12 (d, J = 8.7 Hz, 2H), 7.97 (d, J = 9.0 Hz, 2H), 7.68 (s, 1H), 7.60 (d, J = 8.7 Hz, 2H), 7.52 (d, J = 4.8 Hz, 1H), 7.09 (s, 1H), 4.36 (q, J = 6.6 Hz, 2H), 1.35 (t, J = 7.2, 3H). | m/z 386.3 [M + H]⁺ | ethyl 4-(2-(4-(1H- imidazol-1- yl)phenylamino) pyrimidin-4- yl)benzoate |
| 39 | 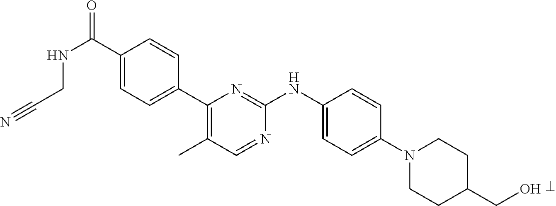 [SMILES: Cc1cnc(Nc2ccc(N3CCC(CO)CC3)cc2)nc1-c1ccc(C(=O)NCC#N)cc1] | 456.23 | ¹H NMR (300 MHz, CDCl₃): δ 8.30 (s, 3H), 7.89 (d, J = 8.2 Hz, 2H), 7.73 (d, J = 8.2 Hz, 2H), 7.48 (d, J = 8.9 Hz, 2H), 6.94 (d, J = 8.6 Hz, 2H), 6.49 (t, J = 6.1 Hz, 1H), 4.43 (d, J = 5.9 Hz, 2H), 3.63 (d, J = 12.0 Hz, 2H), 3.55 (t, J = 5.7 Hz, 2H), 2.72-2.63 (m, 2H), 2.23 (s, 3H), 1.85 (d, J = 13.2 Hz, 2H), 1.48-1.39 (m, 2H), 1.35- 1.31 (m, 1H). | m/z 457.4 [M + H]⁺ | N-(cyanomethyl)- 4-(2-(4-(4- (hydroxymethyl) piperidin-1- yl)phenylamino)- 5- methylpyrimidin- 4-yl)benzamide |
| 40 | 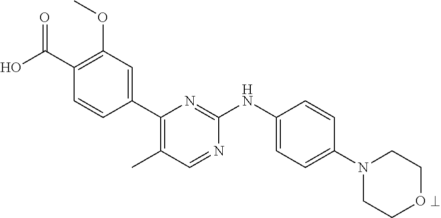 [SMILES: COc1cc(-c2nc(Nc3ccc(N4CCOCC4)cc3)ncc2C)ccc1C(=O)O] | 420.18 | ¹H NMR (300 MHz, d₆- DMSO): δ 9.35 (s, 1H), 8.38 (s, 1H), 7.74 (d, J = 8.1 Hz, 1H), 7.63 (d, J = 9.0 Hz, 2H), 7.36 (s, 1H), 7.26 (d, J = 7.8 Hz, 1H), 6.88 (d, J = 9.3 Hz, 2H), 3.87 (s, 3H), 3.72 (m, 4H), 3.01 (m, 4H), 2.20 (s, 3H). | m/z 421.4 [M + H]⁺ | 2-methoxy-4-(5- methyl-2-(4- morpholinophenyl- amino)pyrimidin- 4-yl)benzoic acid |
| 42 | 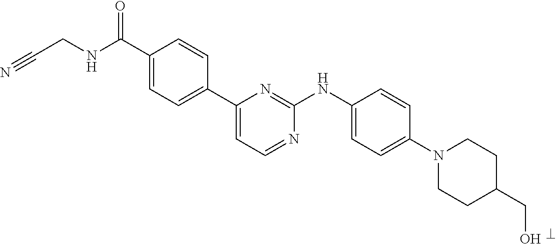 [SMILES: N#CCNC(=O)c1ccc(-c2ccnc(Nc3ccc(N4CCC(CO)CC4)cc3)n2)cc1] | 442.21 | ¹H NMR (300 MHz, CD₃OD): δ 8.44 (d, J = 5.4 Hz, 1H), 8.25 (d, J = 8.4 Hz, 2H), 7.98 (d, J = 9.0 Hz, 2H), 7.60 (d, J = 9.0 Hz, 2H), 7.29 (d, J = 5.4 Hz, 1H), 7.03 (d, J = 9.3 Hz, 2H), 4.53 (brs, 1H), 4.36 (s, 2H), 3.68-3.60 (m, 2H), 3.46 (d, J = 6.3 Hz, 2H), 2.73-2.64 (m, 2H), 1.92-1.82 (m, 2H), 1.68- 1.52 (m, 1H), 1.48-1.32 (m, 2H). | m/z 443.4 [M + H]⁺ | N-(cyanomethyl)- 4-(2-(4-(4- (hydroxymethyl) piperidin-1- yl)phenylamino) pyrimidin-4- yl)benzamide |
| 43 | 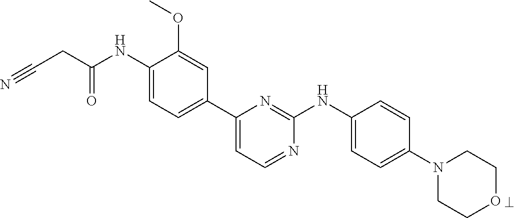 [SMILES: COc1cc(-c2ccnc(Nc3ccc(N4CCOCC4)cc3)n2)ccc1NC(=O)CC#N] | 444.19 | ¹H NMR (300 MHz, d₆- DMSO): δ 9.78 (s, 1H), 9.37 (s, 1H), 8.46 (d, J = 5.1 Hz, 1H), 8.16 (d, J = 8.1 Hz, 1H), 7.85 (d, J = 1.8 Hz, 1H), 7.76 (dd, J = 8.1, 1.8 Hz, 1H), 7.68 (d, J = 9.3 Hz, 2H), 7.35 (d, J = 5.4 Hz, 1H), 6.93 (d, J = 9.0 Hz, 2H), 4.06 (s, 2H), 3.98 (s, 3H), 3.74 (m, 4H), 3.04 (m, 4H). | m/z 445.3 [M + H]⁺ | 2-cyano-N-(2- methoxy-4-(2-(4- morpholinophenyl- amino)pyrimidin- 4- yl)phenyl)acetamide |
| 44 | 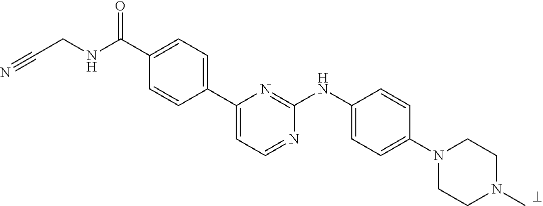 [SMILES: CN1CCN(c2ccc(Nc3nccc(-c4ccc(C(=O)NCC#N)cc4)n3)cc2)CC1] | 427.21 | ¹H NMR (300 MHz, CD₃OD): δ 8.46 (d, J = 5.1 Hz, 1H), 8.25 (d, J = 8.7 Hz, 2H), 7.98 (d, J = 8.7 Hz, 2H), 7.66 (d, J = 9.0 Hz, 2H), 7.31 (d, J = 5.4 Hz, 1H), 7.03 (d, J = 9.0 Hz, 2H), 4.36 (s, 2H), 3.38-3.33 (m, 4H), 3.25-3.20 (m, 4H), 2.80 (s, 3H). | m/z 428.4 [M + H]⁺ | N-(cyanomethyl)- 4-(2-(4-(4- methylpiperazin- 1- yl)phenylamino) pyrimidin-4- yl)benzamide |
| 45 | 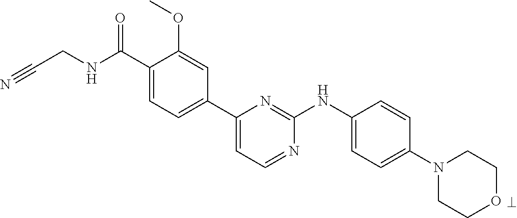 [SMILES: COc1cc(-c2ccnc(Nc3ccc(N4CCOCC4)cc3)n2)ccc1C(=O)NCC#N] | 444.19 | ¹H NMR (300 MHz, d₆- DMSO): δ 9.47 (s, 1H), 8.87 (br t, J = 5.4 Hz, 1H), 8.54 (d, J = 5.1 Hz, 1H), 7.96 (d, J = 8.1 Hz, 1H), 7.90 (s, 1H), 7.82 (d, J = 8.1 Hz, 1H), 7.67 (d, J = 8.7 Hz, 2H), 7.43 (d, J = 5.1 Hz, 1H), 6.93 (d, J = 9.3 Hz, 2H), 4.32 (d, J = 5.4 Hz, 2H), 4.04 (s, 3H), 3.74 (m, 4H), 3.04 (m, 4H). | m/z 445.3 [M + H]⁺ | N-(cyanomethyl)- 2-methoxy-4-(2- (4- morpholinophenyl- amino)pyrimidin- 4-yl)benzamide |
| 46 | 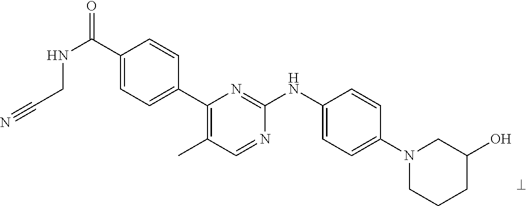 [SMILES: Cc1cnc(Nc2ccc(N3CCCC(O)C3)cc2)nc1-c1ccc(C(=O)NCC#N)cc1] | 442.21 | ¹H NMR (300 MHz, CDCl₃): δ 8.31 (s, 1H), 7.89 (d, J = 8.7 Hz, 2H), 7.72 (d, J = 8.2 Hz, 2H), 7.49 (d, J = 9.3 Hz, 2H), 6.94 (m, 3H), 6.60 (t, J = 5.7 Hz, 1H), 4.42 (d, J = 6.1 Hz, 2H), 3.93 (m, 2H), 3.24-3.20 (m, 2H), 3.07-3.01 (m, 4H), 2.23 (s, 3H), 1.55-2.00 (m, 2H, partially obscured by grease impurity). | m/z 443.3 [M + H]⁺ | N-(cyanomethyl)- 4-(5-methoxy-2- (4- morpholinophenyl- amino)pyrimidin- 4-yl)benzamide |
| 47 | 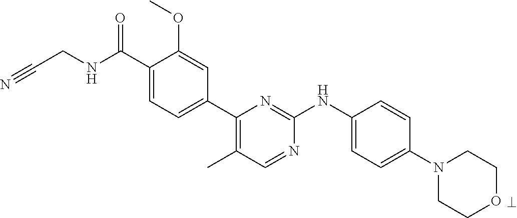 [SMILES: COc1cc(-c2nc(Nc3ccc(N4CCOCC4)cc3)ncc2C)ccc1C(=O)NCC#N] | 458.21 | ¹H NMR (300 MHz, d₆- DMSO): δ 9.33 (s, 1H), 8.87 (t, J = 5.7 Hz, 1H), 8.38 (s, 1H), 7.93 (d, J = 7.8 Hz, 1H), 7.63 (d, J = 9.0 Hz, 2H), 7.41 (d, J = 1.2 Hz, 1H), 7.33 (dd, J = 7.8, 1.5 Hz, 1H), 6.88 (d, J = 9.0 Hz, 2H), 4.32 (d, J = 5.7 Hz, 2H), 3.97 (s, 3H), 3.73 (m, 4H), 3.01 (m, 4H), 2.21 (s, 3H). | m/z 459.3 [M + H]⁺ | N-(cyanomethyl)- 2-methoxy-4-(5- methyl-2-(4- morpholinophenyl- amino)pyrimidin- 4-yl)benzamide |
| 48 | 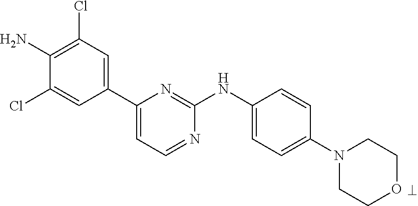 [SMILES: Nc1c(Cl)cc(-c2ccnc(Nc3ccc(N4CCOCC4)cc3)n2)cc1Cl] | 415.10 | ¹H NMR (300 MHz, d₆- DMSO): δ 12.40 (m, 4H), 9.31 (s, 1H), 8.38 (d, J = 5.4 Hz, 1H), 8.08 (s, 2H), 7.62 (d, J = 8.7 Hz, 2H), 7.27 (d, J = 5.4 Hz, 1H), 6.90 (d, J = 9.3 Hz, 2H), 6.07 (brs, 2H), 3.74 (m, 4H), 3.04 (m, 4H), 2.80-2.63 (m, 8H). | m/z 416.2/ 418.2/ 420.2 [M + H]⁺ | 4-(4-amino-3,5- dichlorophenyl)- N-(4- morpholinophenyl) pyrimidin-2- amine.citrate |
| 49 | 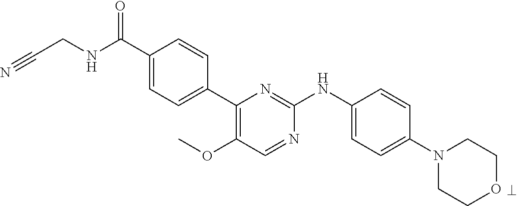 [SMILES: COc1cnc(Nc2ccc(N3CCOCC3)cc2)nc1-c1ccc(C(=O)NCC#N)cc1] | 444.19 | ¹H NMR (300 MHz, CDCl₃): δ 8.25 (s, 1H), 8.22 (d, J = 8.7 Hz, 2H), 7.87 (d, J = 8.6 Hz, 2H), 7.52 (d, J = 9.0 Hz, 2H), 6.93 (d, J = 9.2 Hz, 2H), 6.88 (brs, 1H), 6.45 (t, J = 5.7 Hz, 1H), 4.43 (d, J = 5.8 Hz, 2H), 3.87 (t, J = 4.8 Hz, 4H), 3.87 (s, 3H), 3.12 (t, J = 4.8 Hz, 4H). | m/z 445.3 [M + H]⁺ | N-(cyanomethyl)- 4-(5-methoxy-2- (4- morpholinophenyl- amino)pyrimidin- 4-yl)benzamide |
| 50 | 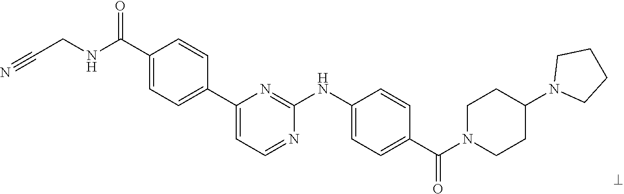 [SMILES: N#CCNC(=O)c1ccc(-c2ccnc(Nc3ccc(C(=O)N4CCC(N5CCCC5)CC4)cc3)n2)cc1] | 509.25 | ¹H NMR (300 MHz, CD₃OD): δ 8.56 (d, J = 5.4 Hz, 1H), 8.29 (d, J = 8.7 Hz, 2H), 8.00 (d, J = 8.7 Hz, 2H), 7.91 (d, J = 9.0 Hz, 2H), 7.45-7.40 (m, 3H), 4.37 (s, 2H), 3.20-2.72 (m, 2H), 2.78-2.72 (m, 4H), 2.56-2.42 (m, 1H), 2.12-1.92 (m, 2H), 1.90-1.84 (m, 5H), 3.60-1.42 (m, 2H), 1.32-1.28 (brs, 1H). | m/z 510.4 [M + H]⁺ | N-(cyanomethyl)- 4-(2-(4-(4- (pyrrolidin-1- yl)piperidine-1- carbonyl)phenyl- amino)pyrimidin-4- yl)benzamide |
| 51 | 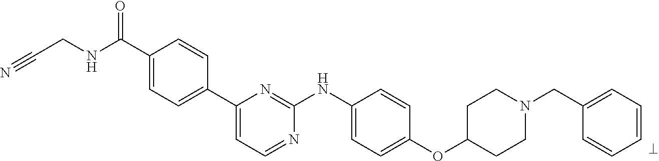 [SMILES: N#CCNC(=O)c1ccc(-c2ccnc(Nc3ccc(OC4CCN(Cc5ccccc5)CC4)cc3)n2)cc1] | 518.24 | ¹H NMR (300 MHz, d₆- DMSO): δ 10.33 (s, 1H), 10.13 (t, J = 5.4 Hz, 1H), 9.35 (d, J = 5.1 Hz, 1H), 9.07 (d, J = 8.7 Hz, 2H), 8.83 (d, J = 8.4 Hz, 2H), 8.49 (d, J = 8.5 Hz, 2H), 8.22 (d, J = 5.1 Hz, 1H), 8.16- 8.02 (m, 5H), 7.73 (d, J = 9.3 Hz, 2H), 5.18 (d, J = 3.6 Hz, 2H), 5.15-5.06 (m, 1 H), 4.30 (s, 2H), 3.55-3.42 (m, 2H), 3.10-2.95 (m, 2H), 2.80-2.67 (m, 2H), 2.50-2.38 (m, 2H). | m/z 519.3 [M + H]⁺ | 4-(2-(4-(1- benzylpiperidin-4- yloxy)phenylamino) pyrimidin-4-yl)- N- (cyanomethyl) benzamide |
| 52 | 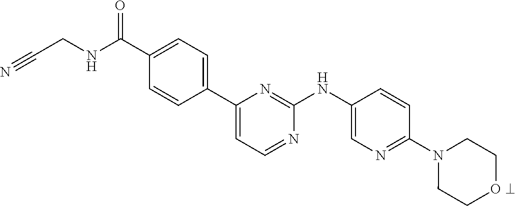 [SMILES: N#CCNC(=O)c1ccc(-c2ccnc(Nc3ccc(N4CCOCC4)nc3)n2)cc1] | 415.18 | ¹H NMR (300 MHz, d₆- DMSO): δ 9.50 (s, 1H), 9.38- 9.32 (m, 1H), 8.54 (d, J = 5.1 Hz, 2H), 8.24 (d, J = 8.4 Hz, 2H), 8.02 (d, J = 8.7 Hz, 2H), 7.97 (dd, J = 9.0, 2.7 Hz, 1H), 7.42 (d, J = 5.4 Hz, 1H), 6.86 (d, J = 9.0 Hz, 1H), 4.35 (d, J = 5.4 Hz, 2H), 3.76-3.68 (m, 4H), 3.40-3.35 (m, 4H). | m/z 416.3 [M + H]⁺ | N-(cyanomethyl)- 4-(2-(6- morpholinopyridin- 3- ylamino)pyrimidin- 4-yl)benzamide |
| 53 | 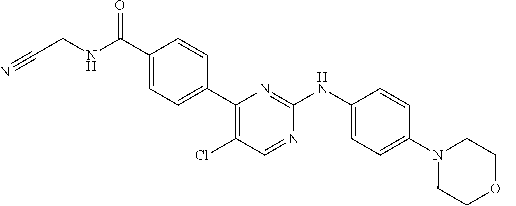 [SMILES: N#CCNC(=O)c1ccc(-c2nc(Nc3ccc(N4CCOCC4)cc3)ncc2Cl)cc1] | 448.14 | ¹H NMR (300 MHz, CDCl₃/CD₃OD): δ 8.41 (s, 1H), 7.98 (m, 4H), 7.56 (d, J = 8.6 Hz, 2H), 6.95 (d, J = 8.6 Hz, 2H), 4.35 (s, 2H), 3.80 (t, J = 4.8 Hz, 4H), 3.08 (t, J = 4.8 Hz, 4H). | m/z 449.3 [M + H]⁺ | 4-(5-chloro-2-(4- morpholinophenyl- amino)pyrimidin- 4-yl)-N- (cyanomethyl) benzamide |
| 54 | 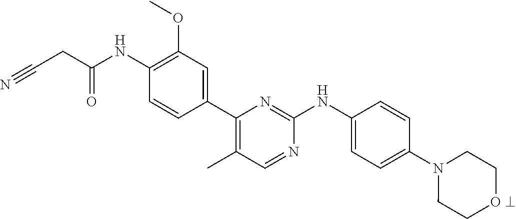 [SMILES: COc1cc(-c2nc(Nc3ccc(N4CCOCC4)cc3)ncc2C)ccc1NC(=O)CC#N] | 458.21 | ¹H NMR (300 MHz, d₆- DMSO): δ 9.74 (s, 1H), 9.25 (s, 1H), 8.34 (s, 1H), 8.10 (d, J = 8.1 Hz, 1H), 7.64 (d, J = 9.0 Hz, 2H), 7.39 (s, 1H), 7.28 (d, J = 8.1 Hz, 1H), 6.87 (d, J = 8.7 Hz, 2H), 4.05 (s, 2H), 3.92 (s, 3H), 3.73 (m, 4H), 3.01 (m, 4H), 2.24 (s, 3H). | m/z 459.4 [M + H]⁺ | 2-cyano-N-(2- methoxy-4-(5- methyl-2-(4- morpholinophenyl- amino)pyrimidin- 4- yl)phenyl)acetamide |
| 55 | 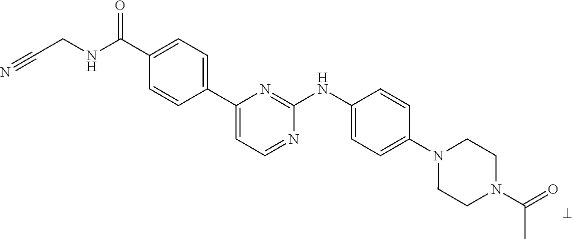 [SMILES: CC(=O)N1CCN(c2ccc(Nc3nccc(-c4ccc(C(=O)NCC#N)cc4)n3)cc2)CC1] | 455.21 | ¹H NMR (300 MHz, d₆- DMSO): δ 9.49 (s, 1H), 9.34- 9.28 (m, 1H), 8.54 (d, J = 5.4 Hz, 1H), 8.26 (d, J = 8.7 Hz, 2H), 8.02 (d, J = 8.7 Hz, 2H), 7.67 (d, J = 9.0 Hz, 2H), 7.41 (d, J = 5.1 Hz, 1H), 6.96 (d, J = 9.0 Hz, 2H), 4.35 (d, J = 5.4 Hz, 2H), 3.62-3.54 (m, 4H), 3.11-3.00 (m, 4H), 2.04 (s, 3H). | m/z 456.3 [M + H]⁺ | 4-(2-(4-(4- acetylpiperazin-1- yl)phenylamino) pyrimidin-4-yl)-N- (cyanomethyl) benzamide |
| 56 | 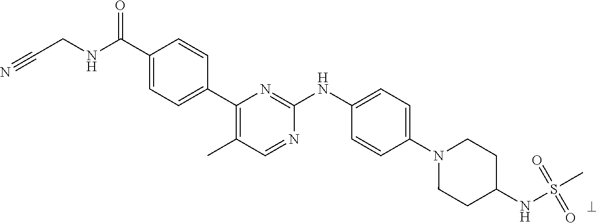 [SMILES: Cc1cnc(Nc2ccc(N3CCC(NS(C)(=O)=O)CC3)cc2)nc1-c1ccc(C(=O)NCC#N)cc1] | 519.21 | | LC- ESI- MS (method B): rt 5.8 min, m/z 520.3 [M + H]⁺ | N-(cyanomethyl)- 4-(5-methyl-2-(4- (4- (methylsulfonamido) piperidin-1- yl)phenylamino) pyrimidin-4- yl)benzamide |
| 57 | 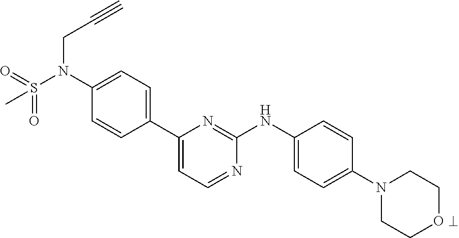 [SMILES: C#CCN(c1ccc(-c2ccnc(Nc3ccc(N4CCOCC4)cc3)n2)cc1)S(C)(=O)=O] | 463.17 | ¹H NMR (300 MHz, d₆- DMSO): δ 9.78 (brs, 1H), 8.55 (d, J = 5.4 Hz, 1H), 8.22 (d, J = 8.7 Hz, 2H), 7.81 (brd, J = 8.7 Hz, 2H), 7.64 (d, J = 8.7 Hz, 2H), 7.48 (d, J = .8 Hz, 2H), 7.44 (d, J = 5.1 Hz, 1H), 7.29 (brd, J = 8.1 Hz, 2H), 7.11 (ap. d, J = 7.8 Hz, 2H), 4.58 (d, J = 2.4 Hz, 2H), 3.86 (m, 4H), 3.41 (t, J = 2.4 Hz, 1H), 3.34 (brm, 4H), 3.13 (s, 3H), 2.28 (s, 3H). | m/z 464.0 [M + H]⁺ | N-(4-(2-(4- morpholinophenyl- amino)pyrimidin- 4-yl)phenyl)-N- (prop-2- ynyl)methane- sulfonamide tosylate |
| 58 | 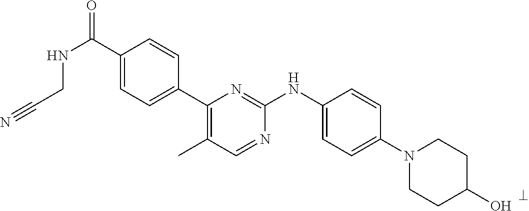 [SMILES: Cc1cnc(Nc2ccc(N3CCC(O)CC3)cc2)nc1-c1ccc(C(=O)NCC#N)cc1] | 442.21 | ¹H NMR (300 MHz, CD₃OD): δ 8.27 (s, 1H), 7.95 (d, J = 8.4 Hz, 2H), 7.75 (d, J = 8.7 Hz, 2H), 7.52 (d, J = 9.1 Hz, 2H), 6.94 (d, J = 9.0 Hz, 2H), 4.34 (s, 2H), 3.78-3.68 (m, 1H), 3.45-3.41 (m, 2H), 2.80-2.75 (m, 2H), 2.21 (s, 3H), 1.93-1.91 (m, 2H), 1.65-1.62 (m, 2H). | m/z 443.3 [M + H]⁺ | N-(cyanomethyl)- 4-(2-(4-(4- hydroxypiperidin- 1- yl)phenylamino)- 5- methylpyrimidin- 4-yl)benzamide |
| 59 | 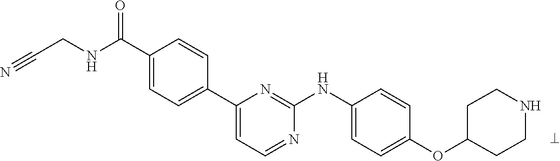 [SMILES: N#CCNC(=O)c1ccc(-c2ccnc(Nc3ccc(OC4CCNCC4)cc3)n2)cc1] | 428.20 | ¹H NMR (300 MHz, d₆- DMSO): δ 9.51 (s, 1H), 9.36- 9.30 (m, 1H), 8.54 (d, J = 5.1 Hz, 1H), 8.26 (d, J = 8.7 Hz, 2H), 8.03 (d, J = 8.4 Hz, 2H), 7.68 (d, J = 9.0 Hz, 2H), 7.41 (d, J = 5.4 Hz, 1H), 6.93 (d, J = 10.2 Hz, 2H), 4.35 (d, J = 5.4 Hz, 2H), 4.32-4.26 (m, 1H), 3.00-1.35 (m, 9H). | m/z 429.3 [M + H]⁺ | N-(cyanomethyl)- 4-(2-(4-(piperidin- 4- yloxy)phenylamino) pyrimidin-4- yl)benzamide |

resin (Agronaut part number 800471) (7.5 g) over 2 days. The solution was filtered, the solids were washed with dichloromethane (2×100 mL), and the filtrate concentrated to give ethyl 4-(2-chloropyrimidin-4-yl)benzoate as an off-white solid (17.73 g, 60%)—additional washing with dichloromethane yielded a further 1.38 g and 0.5 g of product. ¹H NMR (300 MHz, d₆-DMSO)) δ 8.89 (1H, d, J=5.0 Hz); 8.32 (2H, d, J=8.7 Hz); 8.22 (1H, d, J=5.5 Hz): 8.12 (2H, d, J=8.7 Hz); 4.35 (2H, q, J=7.1 Hz); 1.34 (3H, t, J=7.1 Hz); LC-ESI-MS (method B): rt 7.3 min.: m/z 263.0/265.0 [M+H]⁺.

A mixture of ethyl 4-(2-chloropyrimidin-4-yl)benzoate (26.15 g, 99.7 mmol) and 4-morpholinoaniline (23.10 g, 129.6 mmol) was suspended in 1,4-dioxane (250 mL). p-Toluenesulfonic acid monohydrate (17.07 g, 89.73 mmol) was added. The mixture was heated at reflux for 40 h., cooled to ambient temperature, concentrated then the residue was partitioned between ethyl acetate and 1:1 saturated sodium bicarbonate/water (IL total). The organic phase was washed with water (2×100 mL) and concentrated. The aqueous phase was extracted with dichloromethane (3×200 mL). The material which precipitated during this workup was collected by filtration and set aside. The liquid organics were combined, concentrated, triturated with methanol (200 mL) and filtered to yield additional yellow solid. The solids were combined, suspended in methanol (500 mL), allowed to stand overnight then sonicated and filtered. The solids were washed with methanol (2×50 mL) to give, after drying, ethyl 4-(2-(4-morpholinophenylamino)pyrimidin-4-yl)benzoate (35.39 g, 88%). ¹H NMR (300 MHz, d₆-DMSO) δ 9.49 (1H, s); 8.54 (1H, d, J=5.0 Hz); 8.27 (2H, d, J=8.7 Hz); 8.10 (2H, d, J=8.7 Hz), 7.66 (2H, d, J=9.1 Hz); 7.38 (1H, d, J=5.0 Hz); 6.93 (2H, d, J=8.7 Hz); 4.35 (2H, q, J=6.9 Hz), 3.73 (4H, m); 3.04 (4H, m); 1.34 (3H, t, J=6.9 Hz); LC-ESI-MS (method B): rt 7.5 min.; m/z 404.1 [M+H]⁺.

A solution of ethyl 4-(2-(4-morpholinophenylamino)pyrimidin-4-yl)benzoate (35.39 g, 87.6 mmol) in 3:1 methanol/tetrahydrofuran (350 mL) was treated with lithium hydroxide (4.41 g, 183.9 mmol) in water (90 mL). The mixture was heated at reflux for 2 h., cooled, concentrated and acidified with hydrochloric acid (2M, 92.5 mL, 185 mmol). The dark precipitate was filtered, washed with water, and dried under vacuum. The solid was ground to a powder with a mortar and pestle, triturated with methanol (500 ml) then filtered again to yield 4-(2-(4-morpholinophenylamino)pyrimidin-4-yl)benzoic acid as a muddy solid. This material was washed with ether, air dried overnight, and ground to a fine powder with mortar and pestle. On the basis of mass recovery (34.49 g) the yield was assumed to be quantitative. ¹H NMR (300 MHz, d₆-DMSO) δ 9.47 (1H, s); 8.53 (1H, d, J=5.2 Hz); 8.24 (2H, d, J=8.5 Hz); 8.08 (2H, d, J=8.8 Hz), 7.66 (2H, d, J=9.1 Hz); 7.37 (1H, d, J=5.2 Hz); 6.93 (2H, d, J=9.1 Hz); 3.73 (4H, m); 3.04 (4H, m). LC-ESI-MS (method C): rt 7.3 min.; m/z 377.1 [M+H]⁺.

To a suspension of 4-(2-(4-morpholinophenylamino)pyrimidin-4-yl)benzoic acid (theoretically 32.59 g, 86.6 mmol) in DMF (400 mL) was added triethylamine (72.4 mL, 519.6 mmol, 6 eq.) The mixture was sonicated to ensure dissolution. Aminoacetonitrile hydrochloride (16.02 g, 173.2 mmol) was added followed by N-hydroxybenzotriazole (anhydrous, 14.04 g, 103.8 mmol) and 1-ethyl-3-(dimethylaminopropyl) carbodiimide hydrochloride (19.92 g, 103.8 mmol). The suspension was stirred vigorously overnight. The solvent was evaporated under reduced pressure, the residue was diluted with 5% sodium bicarbonate (400 mL) and water (300 mL), giving a yellow solid, which was broken up and filtered. The solids were washed several times with 100 mL portions of water, triturated with hot methanol/dichloromethane (500 mL, 1:1), concentrated to a volume of approximately 300 mL), cooled and filtered. The solids were washed with cold methanol (3×100 mL), ether (200 mL) and hexane (200 mL) prior to drying to afford Compound 3 (31.69 g, 88%). M.p. 238-243° C. Microanalysis: Found C, 66.52; H, 5.41; N, 20.21. C₂₃H₂₆N₆O₁₀S₂ requires C, 66.65; H, 5.35; N, 20.28%. ¹³C NMR (75.5 MHz, d₆-DMSO) δ 166.04, 162.34, 160.26, 159.14, 146.14, 139.87, 134.44, 132.73, 127.80, 126.84, 120.29, 117.49, 115.50, 107.51, 66.06, 49.16, 27.68.

### Example 2—Synthesis of Compound 47

To a solution of 2,4-dichloro-5-methylpyrimidine (244 mg, 1.5 mmol) and methyl 2-methoxy-4-(4,4,5,5-tetramethyl-1,3,2-dioxaborolan-2-yl)benzoate (210 mg, 1.0 mmol) in toluene (3 mL) were added n-propanol (1 mL), aqueous sodium bicarbonate (2 M, 1.5 μL) and tetrakis(triphenylphosphine)palladium[0] (116 mg, 0.1 mmol). The reaction was heated at 110° C. for 40 h, then partitioned between ethyl acetate and saturated aqueous sodium bicarbonate. The aqueous layer was extracted twice further with ethyl acetate and the combined organic fractions were washed with water, brine then dried (sodium sulfate), filtered and concentrated. Silica gel chromatography using 30-60% ethyl acetate/petroleum spirit as eluent provided methyl 4-(2-chloro-5-methylpyrimidin-4-yl)-2-methoxybenzoate as a cream solid (165 mg, 56%); LC-ESI-MS (method B): rt 6.2 min.; m/z 293.3/295.3 [M+H]⁺.

To a solution of methyl 4-(2-chloro-5-methylpyrimidin-4-yl)-2-methoxybenzoate (165 mg, 0.56 mmol) in 1,4-dioxane (5 mL) was added 4-morpholinoaniline (96 mg, 0.54 mmol) and p-toluenesulfonic acid monohydrate (97 mg, 0.51 mmol). The reaction was heated at reflux for 40 h, cooled to room temperature and partitioned between ethyl acetate and saturated aqueous sodium bicarbonate. The aqueous layer was extracted twice more with ethyl acetate and the combined organic fractions were washed twice with 5% aqueous citric acid, water, brine then dried (sodium sulfate) filtered and concentrated to afford the crude product. Trituration with methanol provided methyl 4-(2-(4-morpholinophenylamino)-5-methylpyrimidin-4-yl)-2-methoxybenzoate as a yellow solid (77 mg, 32%); ¹H NMR (300 MHz, d₆-DMSO) δ 9.36 (s, 1H), 8.38 (s, 1H), 7.76 (d, J=8.1 Hz, 1H), 7.62 (d, J=9.0 Hz, 2H), 7.38 (s, 1H), 7.27 (d, J=8.1 Hz, 1H), 6.88 (d, J=9.0 Hz, 2H), 3.89 (s, 3H), 3.82 (s, 3H), 3.72 (m, 4H), 3.01 (m, 4H), 2.12 (s, 3H); LC-ESI-MS (method B): rt 6.7 min.; m/z 435.3 [M+H]⁺.

To a solution of methyl 4-(2-(4-morpholinophenylamino)-5-methylpyrimidin-4-yl)-2-methoxybenzoate (70 mg, 0.16 mmol) in 1,4-dioxane (5 mL) was added aqueous sodium hydroxide (5 M, 5 mL). The reaction was heated at reflux overnight then cooled to room temperature. The yellow solid which precipitated was collected by filtration and washed with water to afford the sodium salt of Compound 40 in quantitative yield.

The sodium salt of 4-(2-(4-morpholinophenylamino)-5-methylpyrimidin-4-yl)-2-methoxybenzoic acid (Compound 40) (0.16 mmol), was acidified by suspending in ethyl acetate and partitioning against 5% aqueous citric acid. Further extraction with ethyl acetate followed by evaporation of the solvent then furnished the free acid which was suspended in dichloromethane (3 mL). To this solution was added triethylamine (111 μL, 0.8 mmol), 1-ethyl-3-(dimethylaminopropyl) carbodiimide hydrochloride (58 mg, 0.3 mmol), aminoacetonitrile hydrochloride (61 mg, 0.4 mmol) and a catalytic amount of N,N-dimethyl aminopyridine. N,N-Dimethyl formamide (2 mL) was added to aid solubility and the reaction was stirred for 64 h. The reaction was incomplete by TLC analysis so O-(7-azabenzotriazol-1-yl)-N,N,N′,N′-tetramethyluronium hexafluorophosphate (76 mg, 0.2 mmol) was added and the reaction stirred for a further 24 h before being partitioned between dichloromethane and saturated aqueous sodium bicarbonate. The aqueous layer was extracted twice further with dichloromethane and the combined organics washed with water, brine then dried (sodium sulfate) filtered and concentrated to afford the crude product. Silica gel chromatography using 0-3% methanol/ethyl acetate as the eluent afforded, as a green/yellow solid, Compound 47 (13.2 mg, 18%).

### Example 3—Synthesis of Compound 90

To a suspension of 4-carboxyphenylboronic acid (5.0 g, 30 mmol) in DMF (5 mL) and dichloromethane (200 mL) at 0° C. was added oxalylchloride (5.9 mL, 66 mmol) dropwise. When gas evolution slowed, the ice bath was removed and the reaction allowed to warm to room temperature over 30 min. The reaction was then heated at 40° C.′ for three hours by which time all solids had dissolved. The dichloromethane was removed by distillation and the DMF solution cooled to 0° C. A solution of aminoacetonitrile hydrochloride (3.05 g, 33 mmol) in DMF (80 mL) and DIPEA (13 mL, 75 mmol) was then added dropwise. After the addition was complete the ice bath was removed and the solution allowed to stir at room temperature for 16 h. Most of the DME was then removed in vacuo and the reaction was partitioned between ethyl acetate and 2 M aqueous hydrochloric acid. The aqueous layer was extracted twice further with ethyl acetate and the combined organic fractions dried (Na₂SO₄) filtered and concentrated under reduced pressure to afford 4-(cyanomethylcarbamoyl)phenylboronic acid as a waxy pale yellow solid (5.34 g, 87%). ¹H NMR (300 MHz, d₆-DMSO)): 9.18 (br. t, J=5.1 Hz, 1H), 7.8-7.9 (m, 4H), 4.31 (d, J=5.4 Hz, 2H); LC-ESI-MS (method B): rt 0.9 min.; m/z 203.3 [M−H]⁻.

To a solution of 2,4-dichloropyrimidine (3.2 g, 0.22 mmol) and 4-(cyanomethylcarbamoyl)phenylboronic acid (3.0 g, 15 mmol) in toluene (146 mL) were added n-propanol (44 mL), aqueous sodium bicarbonate (2M, 22 mL) and tetrakis(triphenylphosphine) palladium[0] (850 mg, 0.7 mmol). The reaction was heated at 90° C.′ for 24 h, then partitioned between ethyl acetate and water. The aqueous layer was extracted twice further with ethyl acetate and the combined organic fractions washed with brine, dried (Na₂SO₄) filtered and concentrated. Silica gel chromatography using 30-70% ethyl acetate/petroleum spirit as eluent provided 4-(2-chloropyrimidin-4-yl)-N-(cyanomethyl)benzamide as a pale yellow waxy solid (1.35 g, 33%). ¹H NMR (300 MHz, d₆-DMSO)) δ 9.40 (t, J=5.4 Hz, 1H), 8.88 (d, J=5.2 Hz, 1H), 8.32 (d, J=8.7 Hz, 2H), 8.23 (d, J=5.1 Hz, 1H), 8.05 (d, J=8.7 Hz, 2H), 4.36 (d, J=5.4 Hz, 2H); LC-ESI-MS (method B): rt 5.3 min.; m/z 273.2/275.2 [M+H]⁺.

A Schlenck flask was dried with a heat gun under vacuum for two minutes and then backfilled at room temperature with nitrogen. Tris(dibenzylideneacetone) dipalladium (9 mg, 0.01 mmol), (2-biphenylyl)di-tert-butylphosphine (5.7 mg, 0.02 mmol), potassium phosphate (56 mg, 0.27 mmol), 4-(2-chloropyrimidin-4-yl)-N-(cyanomethyl)benzamide (52 mg, 0.19 mmol) and 4-[(1,1-dioxidothiomorpholin-4-yl)methyl]aniline (40 mg, 0.17 mmol) were added and mixed together in the flask under a constant flow of nitrogen. The flask was sealed, evacuated under high vacuum and then backfilled with nitrogen. The operation was repeated twice, 1,2-dimethoxyethane (1.9 mL) was added through the rubber septum. The flask was sealed and vigorous stirring was initiated. The mixture was then frozen with liquid nitrogen, degassed under high vacuum and then backfilled with nitrogen (the operation was repeated twice). The sealed flask was then heated to 100° C. overnight. A small amount of tris(dibenzylideneacetone) dipalladium and (2-biphenylyl)di-tert-butylphosphine was added, the mixture was frozen, degassed under high vacuum and backfilled with nitrogen before being heated at 100° C. for a further 16 h. Ethyl acetate was added and the mixture filtered through a sintered funnel. The filtrate was then concentrated and ethyl acetate added. The resulting mixture was then washed with a solution of citric acid (2%) and a saturated solution of sodium chloride. The organic layer was dried (sodium sulfate), filtered and evaporated to give the crude product which was purified by column chromatography using petroleum spirit/ethyl acetate (1/4) to give a residue which was triturated with methanol to give Compound 90 (5.5 mg, 7%).

### Example 4—Synthesis of Compound 73

A round bottomed flask was charged with 4-methanesulfonylaminophenylboronic acid (4.30 g, 20 mmol) and 2,4-dichloropyrimidine (5.97 g, 40 mmol, 2 eq.), toluene (75 mL), n-propanol (25 mL) and aqueous sodium carbonate solution (2M, 18 mL, 1.8 eq.). The reaction mixture was evacuated and backfilled with nitrogen three times before adding tetrakis(triphenylphosphine)palladium(0) catalyst (1.02 g, 4.4 mol %). The reaction mixture was again evacuated and backfilled with nitrogen three times before being heated at 100° C. under a nitrogen atmosphere for 66 hours. The reaction mixture was cooled and stirred at room temperature for several hours during which time the product precipitated from the reaction mixture. The fine yellow solid (3.45 g, 61% yield) was collected by vacuum filtration, washed with methanol and dried under high vacuum. ¹H NMR and LC MS data confirmed this to be the desired N-(4-(2-chloropyrimidin-4-yl)phenyl)methanesulfonamide. ¹H NMR (300 MHz, d₆ DMSO) δ 10.26 (1H, brs); 8.75 (1H, d, J=5.5 Hz); 8.17 (2H, d, J=9.1 Hz); 8.05 (1H, d, J=5.5 Hz); 7.35 (2H, d, J=8.7 Hz); 3.10 (3H, s). LC-ESI-MS (method B): rt 5.5 min.; m/z 284.2/286.1 [M+H]⁺.

N-(4-(2-chloropyrimidin-4-yl)phenyl)methanesulfonamide (750 mg 2.64 mmol) and potassium carbonate (730 mg, 2 eq.) were placed in a round bottomed flask and suspended in acetone (50 mL). The mixture was stirred for several minutes before adding bromoacetonitrile (368 μL, 2 eq.). The reaction mixture was stirred at room temperature for 48 h. The crude reaction mixture was concentrated in vacuo and the residue taken up in ethyl acetate (200 mL) and washed with water (2×100 mL), brine (100 mL) and then dried (sodium sulfate). The organic phase was concentrated in vacuo to give N-(4-(2-chloropyrimidin-4-yl)phenyl)-N-(cyanomethyl)methanesulfonamide (762 mg, 89% yield) as a fawn solid. ¹H NMR (300 MHz, d₆-DMSO) δ 8.86 (1H, d, J=5.0 Hz); 8.28 (2H, d, J=8.7 Hz); 8.18 (1H, d, J=5.5 Hz); 7.66 (2H, d, J=8.7 Hz); 4.97 (2H, s); 3.22 (3H, s). LC-ESI-MS (method B): rt 5.9 min.; m/z 323.2/325.2 [M+H]⁺.

N-(4-(2-chloropyrimidin-4-yl)phenyl)-N-(cyanomethyl)methanesulfonamide (171 mg, 0.53 mmol), 5-amino-2-morpholinobenzoic acid (142 mg, 1.2 eq.) and p-toluene sulfonic acid monohydrate (98 mg, 0.98 eq.) were suspended in 1,4-dioxane (8 mL) and heated at 100° C. overnight. The reaction mixture was cooled to room temperature, concentrated in vacuo. The residue was taken up in ethyl acetate (80 mL) and washed with water (20 mL) and brine (20 mL). The organic phase was then dried and concentrated in vacuo. The residue was repeatedly triturated with methanol (5 mL then 3 mL) to afford, as a cream solid, 5-(4-(4-(N-(cyanomethyl)methylsulfonamido)phenyl)pyrimidin-2-ylamino)-2-morpholinobenzoic acid (101 mg, 37%). ¹H NMR (300 MHz, d₆-DMSO)) δ 17.23 (1H, s) CO₂H; 9.99 (1H, s); 8.74 (1H, d, J=2.7 Hz); 8.62 (1H, d, J=5.0 Hz); 8.33 (2H, d, J=8.7 Hz); 8.01 (1H, dd, J=2.8, J=8.7 Hz); 7.69 (1H, d, J=9.1 Hz); 7.63 (2H, d, J=8.7 Hz); 7.52 (1H, d, J=9.1 Hz); 4.97 (2H, s); 3.81 (4H, m); 3.21 (3H, s); 3.06 (4H, m). LC-ESI-MS (method C): rt 5.4 min.; m/z 509.3 [M+H]⁺.

5-(4-(4-(N-(Cyanomethyl)methylsulfonamido)phenyl)pyrimidin-2-ylamino)-2-morpholinobenzoic acid (50 mg, 0.098 mmol) and O-(7-azabenzotriazol-1-yl)-N,N,N′,N′-tetramethyluronium hexafluorophosphate (41 mg, 1.1 eq.) were dissolved in anhydrous N,N-dimethylformamide (4 mL) and sonicated for 5 minutes. Triethylamine (41 mL, 3 eq.) and N,N-dimethylethylenediamine (21 mL, 2 eq.) were added and the mixture stirred overnight at room temperature. The reaction mixture was then diluted with ethyl acetate (50 mL) and washed with bicarbonate solution (20 mL), water (20 mL) and brine (20 mL). The organic phase was dried (sodium sulfate) and concentrated in vacuo to afford, as a yellow solid, Compound 73 (48 mg, 86% yield).

### Example 5—Synthesis of Compound 65

To a solution of 5-bromo-2,4-dichloropyrimidine (300 mg, 1.3 mmol) in dichloromethane (3 mL) kept at −5° C. was added cold 57% aqueous hydroiodic acid (5 mL). The resulting solution was stirred at −5° C. for 2 hours. Solid sodium carbonate was added in small portions until the solution was pH 7 and the mixture was decolourised by adding 5% aqueous sodium metabisulphite. Water was added until the entire solid dissolved and the organic phase was separated. The aqueous phase was extracted twice with dichloromethane then the combined organic layers were dried over anhydrous sodium sulfate, filtered and concentrated to give crude 5-bromo-2,4-diiodopyrimidine as a white solid (410 mg). This material was used for the next step without further purification. LC-ESI-MS (method B): rt 6.8 min.; m/z 410.9/412.9 [M+H]⁺.

To a mixture of 4-(cyanomethylcarbamoyl)phenylboronic acid (see example 3) (185 mg, 0.9 mmol) and 5-bromo-2,4-diiodopyrimidine (410 mg, 1.0 mmol) in 1,4-dioxane (10 mL), was added 2M aqueous potassium carbonate (100 μL). The resulting mixture was stirred under nitrogen for 5 minutes then tetrakis(triphenylphophine)palladium(0)) (52 mg, 0.045 mmol) was added under a nitrogen atmosphere. The mixture was heated at 80° C. overnight. The cooled reaction mixture was diluted with water and extracted twice with ethyl acetate. The combined organic extracts were washed with water then brine, dried over anhydrous sodium sulfate, filtered and concentrated to give the crude product as a brown solid. The crude material was purified by flash chromatography, eluting with 50% ethyl acetate/petroleum spirit to give 4-(5-bromo-2-iodopyrimidin-4-yl)-N-(cyanomethyl)benzamide (200 mg, 35% over 2 steps). LC-ESI-MS (method B): rt 6.2 min.; m/z 443.0/445.0 [M+H]⁺.

To a round bottom flask containing 4-(5-bromo-2-iodopyrimidin-4-yl)-N-(cyanomethyl)benzamide (45 mg, 0.1 mmol) and 4-morpholinoaniline (27 mg, 0.15 mmol) in 1,4-dioxane (3 mL), was added diisopropylamine (26 mg, 0.2 mmol). The flask was equipped with a reflux condenser and the reaction mixture was heated at reflux overnight. After cooling to room temperature, the reaction mixture was diluted with ethyl acetate, washed with water then brine, dried over anhydrous sodium sulfate and concentrated to give the crude product as a brown solid. The crude material was purified by flash chromatography, eluted with 50% ethyl acetate/petroleum spirit then 80% ethyl acetate/petroleum spirit to give, as a yellow solid, Compound 65 (12 mg, 24%).

### Example 6—Salt Formation from Compound 3

Compound 3 (10.0 g) was suspended in methanol (1 L). Concentrated sulfuric acid (10.52 g, 90% w/w) was added dropwise to the stirring solution. A clear brown solution resulted and a solid lump formed. The solution was filtered quickly then allowed to continue stirring for 3 h (a second precipitate appeared within minutes). After this time the pale yellow precipitate was collected by filtration, washed with methanol (10 mL) then dried under vacuum overnight to afford 4-(4-(4-(4-(cyanomethylcarbamoyl)phenyl)pyrimidin-1-ium-2-ylamino)phenyl) morpholin-4-ium hydrogensulfate, as a pale yellow solid (10.20 g, 69%). m.p. 205° C. Microanalysis: Found C, 45.18; H, 4.36; N, 13.84; S, 10.24. C₂₃H₂₆N₆O₁₀S₂ requires C, 45.24; H, 4.29; N, 13.76; S 10.50%. ¹³H NMR (300 MHz, d₆-DMSO) δ 9.85 (br. s, 1H), 9.34 (t, J=5.4 Hz, 1H), 8.59 (d, J=5.2 Hz, 1H), 8.27 (d, J=8.5 Hz, 2H), 8.03 (d, J=8.5 Hz, 2H), 7.83 (d, J=8.4 Hz, 2H), 7.50 (d, J=5.2 Hz, 1H), 7.34 (br. s, 2H), 4.36 (d, J=5.4 Hz, 2H), 3.89 (br. s, 4H), 3.37 (br. s, 4H); ¹³C NMR (75.5 MHz, d₆-DMSO) δ 166.07, 163.36, 159.20, 158.48, 140.19, 139.34, 136.45, 134.89, 128.00, 127.22, 121.13, 119.89, 117.59, 109.05, 64.02, 54.04, 27.82. LC-ESI-MS (method D): rt 10.0 min.; m/z 415.1 [M+H]⁺.

Compound 3 (0.25 g) was suspended in methanol (25 ml). Methane sulfonic acid (0.255 g) was added dropwise to the stirring solution and a clear brown solution resulted. The solution was allowed to stir for 3 h, after which the volume was reduced to 9 ml. The resultant precipitate was collected and dried under vacuum for 8 h to afford 4-(4-(4-(4-(cyanomethylcarbamoyl)phenyl)pyrimidin-1-ium-2-ylamino)phenyl) morpholin-4-ium methanesulfonate as a pale yellow solid (0.22 g). m.p. 208° C. ¹H NMR (300 MHz, d₆-DMSO)) δ 9.83 (br. s, 1H), 9.35 (t, J=5.3 Hz, 1H), 8.59 (d, J=5.1 Hz, 1H), 8.28 (d, J=8.5 Hz, 2H), 8.04 (d, J=8.5 Hz, 2H), 7.83 (d, J=9.0 Hz, 2H), 7.50 (d, J=5.5 Hz, 1H), 7.31 (d, J=9.0 Hz, 2H), 4.36 (d, J=5.5 Hz, 2H), 3.88 (m, 4H), 3.35 (br. s, 4H), 2.36 (s, 6H); LC-ESI-MS (method D): rt 10.2 min.; m/z 415.3 [M+H]⁺.

Compound 3 (0.50 g) was suspended in methanol (45 ml). A freshly prepared solution of hydrochloric acid in methanol (2.6 ml. HCl cone, 40 mg/ml) was added dropwise to the stirring solution and a clear brown solution resulted. The solution was allowed to stir for 2 h, then the resultant precipitate was collected, washed with methanol (5 ml) and dried under vacuum for 8 h to afford 4-(4-(4-(4-(cyanomethylcarbamoyl)phenyl)pyrimidin-1-ium-2-ylamino)phenyl) morpholin-4-ium chloride a pale yellow solid (0.30 g). m.p. 210° C. ¹H NMR (300 MHz, d₆-DMSO) ¹H NMR (300 MHz, DMSO) δ 9.92 (br. s, 1H), 9.42 (t, J=5.3, 1H), 8.62 (d, J=4.8, 1H), 8.29 (d, J=8.1, 2H), 8.06 (d, J=8.1, 2H), 7.89 (d, J=9.0, 2H), 7.53 (br. s, 3H), 4.36 (d, J=5.4, 2H), 3.82 (br. s, 4H), 3.43 (br. s, 4H). LC-ESI-MS (method D): rt 10.3 min.; m/z 415.3 [M+H]⁺.

### Example 7—Synthesis of Compound 79

A 50 mL two necked round bottom flask was fitted with a magnetic stirrer bar and a dropping funnel. A suspension of NaBH₄ in tetrahydrofuran (100 mg, 2.4 mmol/10 mL) was added, followed by 5-amino-2-morpholinobenzenecarboxylic acid (222 mg, 1.0 mmol) in one portion. A reflux condenser was fitted and the reaction mixture was cooled to 0° C. under nitrogen atmosphere. A solution of iodine in tetrahydrofuran (250 mg, 1.0 mmol/15 mL) was added dropwise to the reaction mixture. After iodine addition was completed and gas evolution had ceased, the reaction mixture was heated at reflux for 6 hours and left stirring at room temperature overnight. Methanol was added slowly until the mixture became clear. The resulting solution stirred at room temperature for 30 minutes then solvent was removed under reduced pressure. The residue was dissolved in 20% KOH (30 mL), stirred for 4 hours and extracted with dichloromethane (3×30 mL). The combined organic extracts were washed with brine, dried over anhydrous Na₂SO₄ and concentrated to give 5-amino-2-morpholinobenzyl alcohol as an off-white solid (150 mg, 72% yield). ¹H NMR (300 MHz, CDCl₃) δ 7.05 (d, J=8.7 Hz, 1H), 6.59 (dd, J=8.4, 2.7 Hz, 1H), 6.49 (d, J=2.7 Hz, 1H), 5.51 (br s, 1H), 4.71 (s, 2H), 3.84 (t, J=5.1 Hz, 4H), 3.60 (br s, 2H), 2.91 (t, J=5.1 Hz, 4H). LC-ESI-MS (method B): rt 2.31 min.; m/z 209.2 [M+H]⁺.

To a suspension of NaH in cold tetrahydrofuran (80 mg/20 mL), 5-amino-2-morpholinobenzyl alcohol (400 mg, 2 mmol) was added. The mixture was stirred for 15 minutes then allyl chloride (150 mg, 2 mmol) and tetrabutylammonium iodide (37 mg, 5 mol %) were added. The resulting mixture was stirred at room temperature for 2 hours then at 60° C. overnight. After cooling to room temperature, water was added (200 μL) and the mixture stirred for 10 minutes then diluted with ethyl acetate. The organic phase was washed sequentially with 10% aqueous ammonium chloride and brine, dried over anhydrous Na₂SO₄, filtered and concentrated to give a yellow solid. The crude product was purified with 50% ethyl acetate in petroleum spirit to obtain 3-((allyloxy)methyl)-4-morpholinobenzenamine as a light orange oil (250 mg, 50% yield). ¹H NMR (300 MHz, CDCl₃) δ 6.95 (d, J=8.3 Hz, 1H), 6.82 (d, J=2.7 Hz, 1H), 6.61 (dd, J=8.2, 2.7 Hz, 1H), 6.10-5.90 (m, 1H), 5.34-5.28 (m, 1H), 5.23 (dd, J=10.5, 1.9 Hz, 1H), 4.57 (s, 2H), 4.07-4.05 (m, 2H), 3.80 (t, J=4.8 Hz, 4H), 3.55 (br s, 2H), 2.83 (t, J=4.6 Hz, 4H). LC-ESI-MS (method B) rt 5.91 min.; m/z 249.3 [M+H]⁺.

3-((allyloxy)methyl)-4-morpholinobenzenamine was converted to Compound 79 by reaction with 4-(2-chloropyrimidin-4-yl)-N-(cyanomethyl)benzamide in the presence of p-toluene sulfonic acid using methods analogous to those described for the synthesis of Compound 3 and Compound 47.

### Compound Analysis

¹H and ¹³C NMR data were acquired on a Brucker AV-300 AVANCE NMR spectrometer. LC-EI-MS and EI-MS

### General Parameters:

LC-EI-MS and EI-MS data were acquired on a Waters 2795 Alliance HPLC coupled to a Waters 2996 Photodiode Array Detector and Integrity TMD Electron Impact Mass Spectrometer operating under control of Waters Millenium³² software version 4.0 with the settings outlined below.

### Mass Spectrometer Parameters:

Helium flow of approximately 0.36 L/min.; acquisition mode set to scan; sampling rate of 1 spectra/sec; source temperature 200° C.; nebuliser temperature 80° C.; expansion region temperature 75° C.; mass range m/z 100-550, m/z 100-650 or m/z 100-700 as required.

### HPLC Parameters

LC-MS parameters were as described for each of the methods outlined below. EI-MS samples were injected and analysed with no column present, with a solvent flow rate of 0.25 mL/min.

### Method A1 (LC-EI-MS)

### Solvent Gradient:

| | | | % (0.5% aq | |
|---|---|---|---|---|
| Time | % MilliQ water | % ACN | formic acid) | Curve |
| 0 | 90 | 0 | 10 | — |
| 0.5 | 90 | 0 | 10 | 6 |
| 7.5 | 0 | 90 | 10 | 6 |
| 10.5 | 0 | 90 | 10 | 6 |
| 11.5 | 90 | 0 | 10 | 6 |
| 14.5 | 90 | 0 | 10 | 6 |

Flow rate: 0.25 mL/min

Column: one of.
Alltima HP C₁₈ 2.1×150 mm, 5 micron
XTerra MS C₁₈, 3.0×100 mm, 3.5 micron
XBridge C₁₈, 3.0×100 mm, 3.5 micron

### Method A2 (LC-EI-MS)

### Solvent Gradient:

| Time | % MilliQ water | % ACN | Curve |
|---|---|---|---|
| 0 | 90 | 10 | — |
| 7 | 0 | 100 | 6 |
| 9 | 0 | 100 | 6 |
| 10 | 90 | 10 | 6 |
| 13 | 90 | 10 | 6 |

Flow rate: 0.25 mL/min

Column: one of
Alltima HP C₁₈ 2.1×150 mm, 5 micron
XTerra MS C₁₈, 3.0×100 mm, 3.5 micron
XBridge C₁₈, 3.0×100 mm, 3.5 micron

### LC-ESI-MS

### General Parameters:

LC-ESI-MS data was acquired on a Waters 2695Xe HPLC coupled to a Waters 2996 Photodiode Array Detector and Waters ZQ Mass Spectrometer operating under electrospray ionization conditions with Masslynx software version 4.1 with the settings outlined below.

### Mass Spectrometer Parameters:

Mass range: m/z 100-650

Scan time: 0.5

Inter scan delay: 0.1

Desolvation gas: 500 L/h N₂

Cone Gas: 100 L/h N₂

Desolvation Temperature: 400° C.

Source Temperature: 120° C.

Cone Voltage: +30 V for ESI positive mode, or
−45 V for ESI negative mode

### HPLC Parameters:

Were one of the following sets of conditions outlined below.

### Method B

### Solvent Gradient:

| Time | % MilliQ water | % ACN | Curve |
|---|---|---|---|
| 0 | 90 | 10 | 1 |
| 5 | 0 | 100 | 6 |
| 6 | 0 | 100 | 6 |
| 7 | 90 | 10 | 6 |
| 10 | 90 | 10 | 6 |

Flow rate: 0.25 ml/min.

Column: XTerra MS C₁₈, 2.1×50 mm, 3.5 micron

### Method C

### Solvent Gradient:

| Time | % MilliQ water | % ACN | % 0.5% formic acid _{(aq)} | Curve |
|---|---|---|---|---|
| 0 | 90 | 0 | 10 | 1 |
| 0.5 | 90 | 0 | 10 | 1 |
| 5.5 | 0 | 90 | 10 | 1 |
| 7.5 | 0 | 90 | 10 | 6 |
| 8.5 | 90 | 0 | 10 | 6 |
| 11.5 | 90 | 0 | 10 | 6 |

Flow rate: 0.25 ml/min.

Column: XTerra MS C18, 2.1×50 mm, 3.5 micron

### Method D

### Solvent Gradient:

| Time | % MilliQ water | % ACN | Curve |
|---|---|---|---|
| 0 | 90 | 10 | 1 |
| 10 | 0 | 100 | 6 |
| 12 | 0 | 100 | 6 |
| 13 | 90 | 10 | 6 |
| 16 | 90 | 10 | 6 |

Flow rate: 0.25 ml/min.

Column: XTerra MS C18, 3.0×100 mm, 3.5 micron

### Example 8—Enzyme Screening

### Compound Dilution

For screening purposes, compounds (in 100% DMSO) were warmed at 37° C.′ for at least 20 minutes before use. A 20 μm stock was initially made in assay buffer, where the final concentration of DMSO was 0.3%. The stocks were then diluted in 384 well Optiplates (Packard) where the final concentration of the compound was 5 μM.

### JAK Tyrosine Kinase Domain Production

JAK kinase domains were produced using the following procedures:

### JAK2

The kinase domain of human JAK2 was amplified from U937 mRNA using the polymerase chain reaction with the following primers:
SALI-jk2 5′-ACG CGT CGA CGG TGC CTI IGA AGA CCG GGA T-3′ [SEQ ID NO. 6]
jk2-NOTI 5′-ATA GTT TAG CGG CCG CTC AGA AIG AAG GTC ATT T-3′ [SEQ ID NO. 7

The JAK2 PCR products were cloned into the pDest20 destination vector (Gibco). The JAK2 plasmid was then transformed into competent DH10Bac cells (Gibco), and the recombinant baculovirus was prepared via Sf9 insect cell transfection.

### JAK3

The kinase domain of human JAK3 was amplified from U937 miRNA using the polymerase chain reaction with the following primers:
XHOI-J3 5′-CCG CTC GAG TAT GCC TGC CAA GAC CCC ACG-3′ [SEQ ID NO. 8]
J3-KPNI 5′-CGG GGT ACC CTA TGA AAA GGA CAG GGA GTG-3′ [SEQ ID NO. 9]

The JAK3 PCR products were cloned into the pDest20 destination expression vector (Gibco). The JAK3 plasmid was then transformed into competent DH10Bac cells (Gibco), and the recombinant baculovirus was prepared via Sf9 insect cell transfection.

### Large Scale Production of Kinase Domains

Baculovirus preparations from each of the JAK family members were infected into one litre of Sf9 (Spodoptera frugiperda) cells (Invitrogen) grown in SF900II serum free medium (Invitrogen) to a cell density of approximately 2×10⁶ cells/ml. Cells were infected with virus at a cell culture to virus stock ratio of 20:1. Cells were harvested and lysed 48 hours post infection. The GST-tagged JAK kinase domains were purified by affinity chromatography on a GSH agarose column (Scientifix).

### Assay Protocols

Kinase assays were performed in 384 well Optiplates (Packard) using an Alphascreen Protein Tyrosine KinaseP100 detection kit The compounds were pre-incubated with affinity purified PTK domain in the presence of phosphotyrosine assay buffer (10 mM HEPES, pH 7.5, 100 mM MgCl₂, 25 mM NaCl, 200 mM sodium vanadate and 0.1% Tween® 20) for 20 minutes. The compounds were then incubated with substrate in the presence of either 80 or 625 um ATP for 60 or 90 minutes. The substrate used was either substrate-1 with the sequence biotin-EGPWLEEEEEAYGWMDF-NH₂ [SEQ ID NO:10] (final concentration 111 μM) or substrate-2 substrate with the sequence biotin-EQEDEPEGDYFEWLEPE [SEQ ID NO: 16] (final concentration 133 μM). Alphascreen phosphotyrosine acceptor beads followed by streptavidin donor beads at a concentration of 1/100 in stop buffer were added to each well under subdued light and incubated for 2-3 hours. The Alphascreen plates were read on a Packard Fusion Alpha instrument

The enzyme assay results and structural data for selected compounds is given below in Table 2, where +++ is <100 nM, ++ is <500 nM and + is <1 μM

### Example 9—Cellular Screening

### Compound Dilution

For screening purposes, compounds were diluted in 96 well plates at a concentration of 20 μM. Plates were warmed at 37° C. for 30 minutes before the assay was performed.

### Establishment of the TEL:JAK2 Cell Line

The coding region encompassing nucleotides 1-487 of TEL was amplified by PCR using the oligonucleotides STEL (5′-GGA GGA TCC TGA TCT CTC TCG CTG TGA GAC-3) [SEQ ID NO 11] and 3TEL (5′-AGGC GTC GAC TTC TTC TTC ATG GTT CTG-3′) [SEQ ID NO 12] and U937 mRNA as a template. A BamHI restriction site was incorporated into the STEL primer, and a Sal I restriction site was incorporated into the 3TEL primer. The regions encompassing the kinase domain of JAK2 (nucleotides 2994-3914; JAK2F 5′-ACGC GTC GAC GGT GCC TTT GAA GAC CGG GAT-3′ [SEQ ID NO 13]; JAK2R 5′-ATA GTT TAG CGG CCG CTC AGA ATG AAG GTC ATT T-3′) [SEQ ID NO 14] and JAK3 (nucleotides 2520-3469; JAK3F 5′-GAA GTC GAC TAT GCC TGC CAA GAC CCC ACG ATC TT-3′) [SEQ ID NO 15] were generated by PCR using Taq DNA polymerase (Gibco/BRL) and U937 miRNA as a template. A Sal I restriction site was incorporated into the forward primer of JAK2 and JAK3, a Not I site was incorporated into the JAK2 reverse primer and a Xba I site was added to the reverse primer of JAK3.

A TEL/Jak2 fusion was generated by digestion of the TELPCR product with BamH I/Sal I restriction enzymes, digestion of the JAK2 PCR product with Sal I/Not I restriction enzymes, followed by ligation and subcloning of the ligation product into the mammalian expression Vector pTRE 2 (Clontech), which was prepared by digestion with BamH I-Not I restriction enzymes, to give the the TEL/Jak2 fusion plasmid pTELJAK2.

The TEL/Jak3 fusion was prepared by ligation of the JAK3 Sal I/Not I cleaved kinase domain PCR product with the BamH I/Sal I restriction digested TEL product, followed by ligation of the ligation product into the BamH I/Not I digested pTRE2, to give the TEL/Jak3 fusion plasmid pTELJAK3.

The growth factor dependent myelomonocytic cell line BaF3 bearing the pTET-off plasmid (Clontech) was transfected with either pTELJAK2 or pTELJAK3, and the transfected cells were selected for growth-factor independent cell growth. The BaF3 wild-type cells were cultured in DMEM containing 10% FCS, 10% WEHI 3B conditioned medium. The BaF3 TELJAK cells (BafT_J2 or BafT_J2) were cultured in DMEM 10% Tet-System Approved FBS (without WEHI 3B conditioned medium).

Cellular assays were performed as follows:
Cell suspensions were prepared by harvesting cells from culture (the cells used in this test were in late log phase growth with high viability.) Cells were diluted in the appropriate growth medium, as described above, to 1.1× final concentration (from 50,000 cell/mL to 200,000 cell/mL, depending on cell line).

Compounds to be tested were added (10 μL, 10× final concentration) to a flat bottomed 96-well plate. The cellular suspension (90 μL per well) was then added, and the plate incubated for 48-72 hr at 37° C., 5% CO₂. Alamar Blue 10 μL per well was added and the plates returned to the incubator for a further 4-6 hours. The plates were then read at 544 nm.

### Results

Result are given in table 2 where +++ is <1 μM, ++ is <5 μM and + is <20 μM

TABLE 2

| Compound | | | | | |
|---|---|---|---|---|---|
| No. | JAK2_IC50_nM | JAK3_ IC50_nM | BafT_J2_IC50_μM | BAF3wt_IC50_μM | CTLL2_IC50_μM |
| 1 | +++ | | >20 | >20 | >20 |
| 3 | +++ | ++ | +++ | ++ | ++ |
| 5 | ++ | | >20 | NT | NT |
| 6 | + | | >20 | NT | NT |
| 7 | >1000 | | | | |
| 8 | ++ | | | | |
| 9 | ++ | ++ | ++ | ++ | ++ |
| 11 | +++ | | ++ | ++ | ++ |
| 16 | >1000 | >1000 | >20 | >20 | >20 |
| 17 | ++ | + | ++ | + | >20 |
| 18 | ++ | + | ++ | >20 | + |
| 19 | >1000 | >1000 | >20 | | |
| 20 | +++ | +++ | +++ | +++ | ++ |
| 21 | ++ | ++ | + | + | + |
| 22 | >1000 | ++ | >20 | | |
| 23 | + | ++ | ++ | | |
| 25 | +++ | ++ | ++ | ++ | ++ |
| 31 | +++ | ++ | ++ | ++ | ++ |
| 34 | +++ | ++ | + | ++ | ++ |
| 36 | +++ | +++ | +++ | ++ | ++ |
| 39 | +++ | +++ | +++ | ++ | ++ |
| 42 | +++ | ++ | ++ | ++ | ++ |
| 44 | +++ | ++ | +++ | ++ | ++ |
| 45 | +++ | +++ | +++ | +++ | +++ |
| 46 | +++ | +++ | +++ | ++ | +++ |
| 50 | +++ | + | ++ | + | >20 |
| 53 | +++ | ++ | +++ | ++ | ++ |
| 55 | +++ | +++ | +++ | ++ | ++ |
| 56 | +++ | +++ | +++ | ++ | ++ |
| 57 | +++ | + | ++ | ++ | ++ |
| 58 | +++ | +++ | +++ | ++ | +++ |
| 59 | +++ | ++ | ++ | +++ | ++ |
| 65 | +++ | +++ | +++ | +++ | +++ |
| 75 | +++ | + | ++ | ++ | ++ |
| 76 | +++ | + | ++ | ++ | ++ |
| 79 | +++ | ++ | +++ | +++ | ++ |
| 80 | +++ | ++ | +++ | ++ | ++ |
| 81 | +++ | >1000 | +++ | ++ | ++ |
| 82 | +++ | >1000 | +++ | ++ | ++ |
| 85 | +++ | +++ | +++ | ++ | +++ |
| 89 | +++ | ++ | ++ | ++ | ++ |
| 90 | +++ | ++ | ++ | +++ | + |
| 92 | +++ | +++ | +++ | +++ | +++ |
| 93 | +++ | ++ | +++ | +++ | +++ |

(NT = Not Tested)

### Example 10—Fluorescence Activated Cell Sorter (FACS)

### Multiparameter Intracellular Flow Cytometric Analysis of STAT 5 Phosphorylation

The human erythroleukemic cell line, HEL 92.1.7 (ATCC, TIB-180), was grown in RPMI 1640 containing 10% FCS supplemented with 1 mM sodium pyruvate. For phosphor-STAT 5 determination, HEL cells were grown in RPMI 1640+1% PCS for 18 hours at 37° C. and 2×10⁵ cells per assay point were exposed to DMSO/test compounds for 2 hours at 37° C. The cells were centrifuged at 1300 rpm for 3 minutes and fixed in paraformaldehyde (2% final concentration) for 15 minutes at 37° C. After centrifugation, cells were permeabilized in 90% methanol at 4° C. for 30 minutes. Following three washes in PBS-2% FCS, the staining was performed as follows using BD PharMingen phycoerythrin-conjugated mouse immunoglobulin isotype control (Cat. No. 551436 and phycoerythrin-conjugated mouse Ig (antibody to STAT 5 (Y694) (Cat. No. 612567).

Staining proceeded for 1 hour at room temperature in the dark, followed by 3 washes in PBS-2% FCS. The cells were next resuspended in 800 μL PBS-FCS for FACS analysis. Flow cytometry was performed using a Beckman Cell Lab Quanta SC System with 3 colour and side scatter capabilities. Data analysis was performed with CXP analysis software (version 2.2). The median fluorescence intensity (MFI) was used to determine fold change upon treatment of cells with specific inhibitor compounds, calculated as the MFI_{stimulated}/MFI_{unstimulated} ratio for the phosphospecific antibody fluorescence channel (FL2).

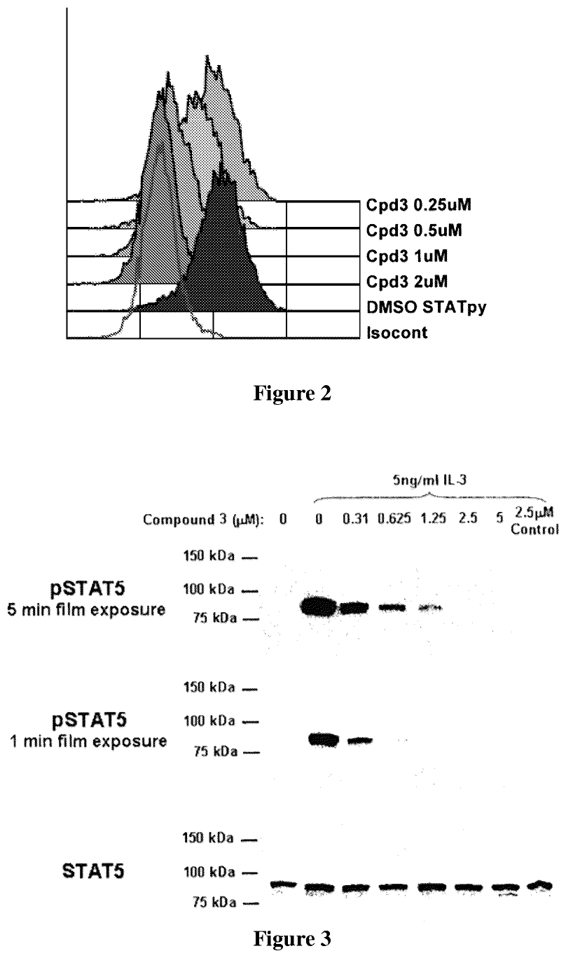

The results shown in FIG. 2 clearly show a dose-dependent effect on STAT5 phosphorylation by treatment with compound 3.

### Example 11—Western Blots

### Experiment 1

### Methodology

The murine pro-B cell line BaF3 was routinely maintained in RPMI 1640 media containing 10% FCS. On the day of the experiment, cells were washed twice in PBS, and resuspended in RPMI 1640 media containing 0.1% FCS. After 2 hours of serum deprivation, cells were treated with the desired concentration of Compound 3, Control Compound, or vehicle alone (DMSO) for a further 2 hours. Mouse IL-3 was then added to cells at a final concentration of 5 ng/ml for 15 minutes. Cells were then placed on ice and washed twice in ice-cold PBS. Washed cell pellets were snap-frozen in liquid nitrogen and stored at −80° C.

Cell pellets were lysed on ice in RIPA buffer, and lysates clarified by centrifugation (20,000×g, 4° C., 5 min). The protein concentration of lysates was determined by the Bradford method, and equal amounts of protein (60 μg/lane) were separated by SDS-PAGE. Protein was then transferred to PVDF, and Western blotting performed using an antibody that specifically recognizes STAT5 phosphorylated at tyrosine 694. The membrane was then stripped and reprobed with an antibody that recognizes total STAT5 protein.

The results shown in FIG. 3 clearly show a dose-dependent effect on STAT5 phosphorylation by treatment with compound 3.

### Experiment 2

### Methodology

The human erythroleukemic cell line HEL 92.1.7 was routinely maintained in RPMI 1640 media containing 10% FCS. The day before the experiment, cells were washed twice in PBS, resuspended in RPMI 1640 media containing 1% FCS, and cultured overnight.

The following day, cells were treated with the desired concentration of Compound 3, Control Compound, or vehicle alone (DMSO)) for 2 hours. Cells were then placed on ice and washed twice in ice-cold PBS. Washed cell pellets were snap-frozen in liquid nitrogen and stored at −80° C.

Cell pellets were lysed on ice in RIPA buffer, and lysates clarified by centrifugation (20,000×g, 4° C., 5 min). The protein concentration of lysates was determined by the Bradford method, and equal amounts of protein (60 μg/lane) were separated by SDS-PAGE. Protein was then transferred to PVDF, and Western blotting performed using an antibody that specifically recognizes STAT5 phosphorylated at tyrosine 694. The membrane was then stripped and reprobed with an antibody that recognizes total STAT5 protein.

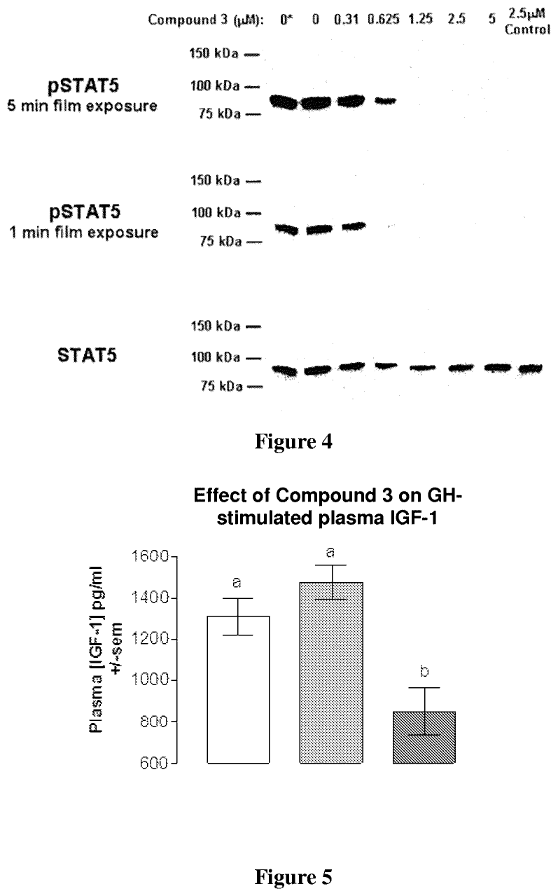

The results shown in FIG. 4 show a decrease in STAT5 phosphorylation upon treatment with compound 3.

### Example 12—Efficacy of Compound 3 on JAK2-Dependent Physiology and Tumour Cell Growth

The effect of Compound 3 on growth hormone—stimulated insulin—like growth factor—1 concentrations in mouse plasma.

Circulating IGF-1 concentrations (mean±s.e.m.) in female C3/H mice (n=6/group) after administration of compound 3 (50 mg/kg), or vehicle only (Control, +GH), by oral gavage 8 h and 30 min prior to subcutaneous administration of growth hormone (+GH, 30 μg/mouse) or saline (Control) at time 0. Blood samples were collected 6 h post-GH administration, and plasma IGF-1 concentrations measured using an ELISA for mouse IGF-1 (R & D Systems).

Different superscripts denote significant differences (p<0.05) between groups detected by one—way ANOVA and Bonferroni's test post-hoc.

The results shown in FIG. 5 show a marked decrease in plasma IGF-1 concentration after treatment with compound 3.

Efficacy of orally administered Compound 3 in a subcutaneous tumour model of Ba/F3 TelJAK2 cells in nude mice.

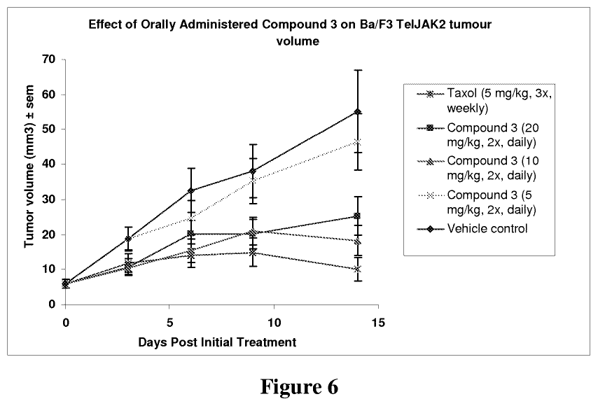

Balb/C^{nu/nu} mice were inoculated subcutaneously with mouse Ba/F3 TelJAK2 cells (2.5×10⁶/mouse), and dosing b.i.d. by oral gavage with compound 3 (20 mg/kg, 10 mg/kg, or 5 mg/kg), or vehicle only (5% N-methylpyrrolidone, 0.1 M Captisol®), or Taxol® (5 mg/kg i.v. 3× weekly, n=15 mice/group). Dosing commenced 11 days post-tumour cell inoculation, when tumours were palpable (mean tumour volume of 6 mm³). Tumour dimensions were measured twice weekly. By dosing day 14, the mean percentage T/C values were 39% for compound 3 at 20 mg/kg b.i.d., 25% at 10 mg/kg b.i.d., and 82% at 5 mg/kg/day. Comparison of tumour volumes after 14 days of dosing by t-test (Mann Whitney Rank Sums Tests) found smaller tumour volumes (p<0.05) in groups treated with compound 3 at 20 mg/kg b.i.d., and 10 mg/kg b.i.d., and Taxol, compared to the Vehicle Control Treated Group and the 5 mg/kg Compound 3 Treated Group, which were not different from each other. A more stringent statistical test (Kruskal Wallis One way ANOVA) followed by Dunn's multiple comparison against the Control Group post-hoc identified a significant difference (p<0.05) between the Vehicle Control Treated Group and either the Compound 3 Treated Group (either 10 or 20 mg/kg b.i.d.) or the Taxol treated Group. The results are shown in FIG. 6.

The results show that compound 3 inhibits the JAK2 enzyme in vitro, as well as the in
vitro growth of Baf3Tcl Jak2 cells, which are dependent on constitutively active Jak2 for growth and survival. Baf3Tcl JAK2 cells growing in vivo as a tumour, are also inhibited by compound 3 in a dose-dependent manner. In addition the results demonstrate that compound 3 inhibits growth hormone (and therefore JAK2 dependent)—driven IGF-1 synthesis and secretion from the mouse liver in vivo.

### Example 13—Additional Compound Evaluation

The compounds can also be tested in a murine model of JAK2^{V617F}-positive myeloproliferative disease (MPD)

### Establishment of JAK2^{V617F}-Positive MPD

Bone marrow from male 5-Flurouracil-treated Balb/c mice could be infected with a JAK2-V617F—GFP retrovirus and retroorbitally injected into lethally irradiated female recipients. From day 21 on the mice could be monitored by daily inspection and twice weekly blood counts+FACS for GFP-positive cells. It would be expected that a rise in hematocrit could occur around day 28 and a rise of the white blood cell count around day 40.

Treatment with Compounds

Early intervention group: Treatment would start on day 21 with compound or carrier given per oral gavage (12 mice in each group). Mice could be monitored by daily inspection and twice weekly blood counts+FACS for GFP-positive cells. Animals would be sacrificed on day 60 8-12 h after the last drug dose. Moribund mice or mice with a white cell count over 200,000/nl or weight loss >20% could be sacrificed earlier.

Late intervention group: Groups of 3 mice could be sacrificed on day 29, 36, 43, 50 and 57 and bone marrow and spleen could be analyzed for reticulin fibrosis. Treatment could start with compound or carrier given per oral gavage as soon as fibrosis is documented in 3/3 mice. Mice could be monitored by daily inspection and twice weekly blood counts+FACS for GFP-positive cells. Animals could be sacrificed after 30 days of therapy 8-12 h after the last drug dose. Moribund mice or mice with a white cell count over 200,000/nl or weight loss >20% could be sacrificed earlier. Animals could be subjected to necropsy.

### Analysis of Tissues and Survival

Liver and spleen weights could be determined. Tissue sections from bone marrow, liver and spleen could be analyzed by HE stain. Marrow and spleens could also be silver-stained to assess reticulin fibrosis. Spleen and marrow cells could be analyzed by FACS for GFP, lineage markers, JAK2 and STAT5 phosphorylation. Blood could be collected by heart puncture and plasma separated and frozen for drug concentration measurement. Survival between groups could be compared with the Kaplan-Meyer method.

Assessment of the activity of JAK2 inhibitors in colony-forming assays of human hematopoietic cells

Peripheral blood mononuclear cells from patients with MPD (predominantly myelofibrosis) with and without JAK2^{V617F} mutation (N=10 for each) and 5 normal controls (commercial supplier) could be isolated by density gradient centrifugation (Ficoll). CD34+ cells can be selected using commercial kits to enrich for progenitor cells. CD34+ cells can be plated in triplicate in methylcellulose supplemented with fetal bovine serum and cytokines (+/− EPO). After incubation of the plates for 2 weeks erythroid and myeloid colony formation could be assessed under an inverted microscope.

### Cancer

The effect of the compounds on tumor initiation, progression and metastasis can be evaluated in relevant in vivo animal efficacy models. Models could be human tumor xenografts models in immuno-deficient mice, from human tumor cell lines or preferably from primary or metastatic human tumors. Other models might be human tumor xenografts grown in orthotopic sites, models of disseminated disease and transgenic or labeled tumors models. Models could also include surgical resection of primary tumor and evaluation of metastatic disease.

Models could be selected to ensure that the molecular drug targeted is expressed. Examples of tumors displaying deregulation of the JAK/STAT pathway include prostate carcinoma, breast cancer, colon carcinoma, including leukemia, lymphoma, myeloma, ovarian tumors, melanoma, lung carcinoma, glioma, renal-cell tumors.

Efficacy can be measured in these models by various outcomes depending on tumor type (solid, leukemia or metastatic) and might include measure of tumor onset, tumor growth rate, tumor burden, tumor growth delay, tumor cell kill, incidence of metastasis, imaging of tumor and invasiveness/metastasis by various approaches including labeled cells or reagents, survival, angiogenesis, histopathology.

The in vivo animal efficacy models might also be used for determination of the additivity or synergy of the effect of the compounds in combination with other drugs,

Asthma is restricted to human species, but animal models are often used to investigate particular aspects of this human disease. Bronchial biopsies and bronchoalveolar lavage (BAL) fluid recovered from patients with asthma have been shown to contain an increased number of activated T cells, B cells, eosinophils and mast cells. Many patients with asthma are sensitized and have specific immunogloulin E (IgE) antibodies to one or more inhalant allergens. Atopy is, considered to be a major cause of asthma. In atopic individuals, inhalation of allergens preferentially induces a T-helper 2 cell (Th2) response. In the majority of current models, mice are sensitized by intraperitoneal (ip) injection of ovalbumin (OVA), often together with a Th2 skewed adjuvant, such as alum. In the classical mouse model for asthma, C57/BL6 mice are actively sensitized on day 0 by ip injection of 10 μg of OVA absorbed onto 1 mg of alum. From day 14-21 the mice are exposed daily to aerosolized OVA over a 30 minute period. On day 22, airway inflammation is apparent. BAL fluid recovered from these animals demonstrate an increase in peri-bronchiolar space consisting of mixed cellular infiltrates of mononuclear cells and eosinophils. OVA-specific IgE antibodies can be demonstrated in the serum of sensitized animals. The mononuclear cell population consists mainly of cells of Th2 phenotype secreting cytokines IL-4 and IL-5. IL-4 promotes isotype switching of B cells towards IgE synthesis and IL-5 influences the production, maturation and activation of eosinophils.

### PAH

The compounds of formula I can be tested in the dog model of pulmonary hypertension as described in Gust, R and Schuster, D. P. Experimental Lung Research, 27:1-12, 2001. They can also be tested in a rabbit model of monocrotaline induced pulmonary hypertension. The compounds of formula I can also be tested in humans with pulmonary arterial hypertension. The effect of the compounds of formula I can be tested in humans with pulmonary arterial hypertension by measurement of its acute effects on cardiopulmonary hemodynamics. The effect of the compounds on right ventricular pressures, pulmonary artery pressures, pulmonary vascular resistance, and cardiac output may be determined. The effect of the compounds on the six minute walk time, and maximal oxygen consumption may be determined in humans with P AH. The effect of the compounds on quality of life (as measured by a questionnaire), hospitalization, and survival may be determined in humans with PAH. In humans PAH may be caused by genetic abnormalities (i.e., primary or familial PAH) or secondary causes such as scleroderma, uncorrected congenital heart disease, mixed collagen vascular disorder, hepatitis C, or other liver disease, HIV infection, or hereditary hemorrhagic teleangiectasia. The effect of the compounds may also be tested on human endothelial cells, fibroblasts and/or smooth muscle cell lines: for example, determination of IC50 for STAT3 phosphorylation in human pulmonary artery smooth muscle cell lines. Cell lines from other species, i.e., the rat may also be examined. The effect of the compounds on precontracted vascular rings from human blood vessels, or blood vessels from other species, i.e., the rat, may be examined. For example, rat pulmonary artery rings preconstricted with phenylephrine, or endothelin, or serotonin, or vasopressin, angiotensin II, or KCL may be studied to determine the dose response to the compounds for vasorelaxation. Other vasoconstrictors may be examined.

The effect of the compounds on hypoxia induced pulmonary vasoconstriction may be examined. A model of hypoxia induced pulmonary hypertension might include study of rats, such as the Fawn-Hooded rat exposed to low oxygen (i.e., 5 percent oxygen). Another model of hypoxia induced pulmonary hypertension might include the fetal calf maintained in a high altitude chamber.

The effect of the compounds may be examined in transgenic models of pulmonary hypertension: i.e., the BMPR2 knockout mouse treated with IL6, the caveolin1 knock out mouse, or the vasoactive intestinal peptide knockout mouse.

The effect of the compounds on histopathologie changes that occur in both human and animal models of PAH may be measured. For example, the compounds may decrease the extent of plexiform lesions in the pulmonary arterioles of diseased lungs. The plexiform lesion consists of endothelial cells, smooth muscle cells, and fibroblasts which proliferate and obstruct to a varying degree, the pulmonary arteriolar lumen.

### Example 14—Ex Vivo Analysis of Compound 3 in Cells from JAK2V617F Positive Patients

To assess the activity of small molecule inhibitors of JAK2 an assay has been developed to quantify the activity of the JAK-STAT pathway by measuring the phosphorylation status of the downstream protein STAT5. After ligand binding, a haemopoietic cytokine receptor undergoes conformational change activating associated JAK2 protein. Activated JAK2 then phosphorylates the intracellular portion of the receptor forming binding sites for the recruitment of intracellular signaling proteins. STAT5 is one protein that is recruited to the activated cytokine receptor complex, where it is phosphorylated and then translocates to the nucleus to regulate the expression of a suite of genes that mediate cellular growth and differentiation.

Intracellular flow cytometry can be used to measure tyrosine phosphorylated STAT5 (pYSTAT5) in specific cell populations by gating on lineage-specific haemopoietic surface markers. This is particularly important for JAK2 V617F positive myeloproliferative disease as the clone containing the mutation only forms a variable fraction of all haemopoietic cells within the bone marrow. Erythroid cells have been selected for examination in this study as this lineage is hyperplastic in PV.

### Methods

Bone marrow was collected from the ileal crest of patients with JAK2 V617F positive myeloproliferative disease. Flow cytometry assays were performed on fresh bone marrow samples on the day of the biopsy procedure. Bone marrow mononuclear cells were collected by density gradient centrifugation and then 0.75-1.0×10⁶ cells were incubated with compound 3 at various concentrations for one hour in indicator-free RPMI at 37° C. Cells were maximally stimulated with erythropoietin for 10 minutes and then fixed by adding 4% formaldehyde directly into the culture medium. Cells were then permeabilised by cold methanol and then optimal concentrations of fluorescent-labeled antibodies added. Erythroid cells were selected for measurement of pYSTAT5 based on cell surface protein expression (CD45^{lo}, CD71^{hi} population).

### Results

Compound 3 was tested in the erythroid cell population at varying concentrations from 3 μM to 0.0041 μM. The first bone marrow specimen was examined with a concentration range of inhibitors from 3 μM to 0.037 μM. The next two patient specimens were examined with a concentration range of between 1 μM and 0.0041 μM.

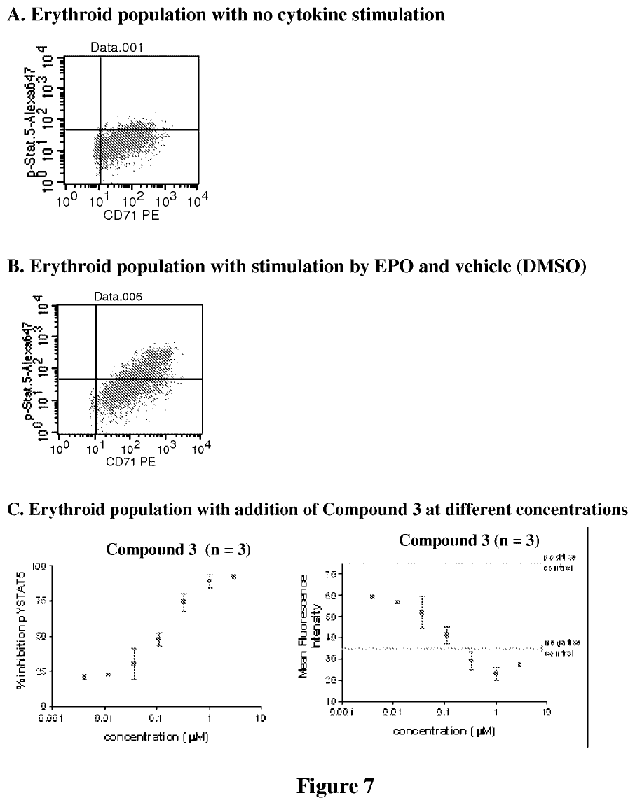

Unstimulated bone marrow samples with no inhibitor (FIG. 7A—the negative control) showed a variable amount of baseline pYSTAT5 phosphorylation from 6 to 32% of the total gated erythroid population. Erythropoietin (EPO) stimulation increased the pYSTAT5 activity in erythroid cells in all specimens examined. This increase in pYSTAT5 with stimulation was most apparent in the subset of cells with the highest CD 71 expression (FIG. 7B), consistent with activation of the more immature cells within the erythroid population.

All patient samples demonstrated a dose-dependent reduction in STAT5 phosphorylation with increasing dose of inhibitor. Results of flow cytometry experiments are presented in two different formats (FIG. 7C). The pYSTAT5 positive population is quantitated as the percentage of cells in the upper right quadrant of the dot plot graphs (FIGS. 7A and B). The threshold for pYSTAT5 positive events in these graphs is based on the isotype control antibody staining and was consistent between experiments. Only a subset of the total erythroid population became positive with EPO stimulation and this was maximal in the positive control sample. As the dose of inhibitor was increased the number of pYSTAT5 positive events decreased and this is presented as the percentage of pYSTAT5 positive events compared to the pYSTAT5 positive events in the positive control in the left panel of FIG. 7C. This is a relative measurement within each individual patient specimen.

The second format of presentation is presented in the right panel of FIG. 7C. This measurement represents the mean fluorescence intensity in the pYSTAT5 channel and includes both erythroid cells that are stimulated by EPO and those that are not. This is an absolute value measurement of fluorescence and there was variability between these values between the three individuals tested. As the concentration of inhibitor is decreased the mean fluorescence of the total erythroid population moved towards the value of the positive control.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

What is claimed is:

1. A method for the treatment of myelofibrosis in a mammalian subject, the method comprising:
administering to a mammalian subject in need thereof one or more tablets comprising an effective amount of a compound of the following formula: 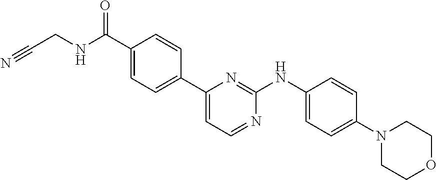 [SMILES: N#CCNC(=O)c1ccc(-c2ccnc(Nc3ccc(N4CCOCC4)cc3)n2)cc1]
or an enantiomer thereof, a prodrug thereof, or a pharmaceutically acceptable salt thereof.

2. The method of claim 1, wherein the one or more tablets contain the compound in admixture with non-toxic pharmaceutically acceptable excipients.

3. The method of claim 1, wherein the myelofibrosis is primary myelofibrosis.

4. The method of claim 1, wherein the mammalian subject is a human subject.

5. A method for the treatment of myelofibrosis in a human subject, the method comprising:
administering to a human subject in need thereof one or more tablets comprising an effective amount of a compound of the following formula:

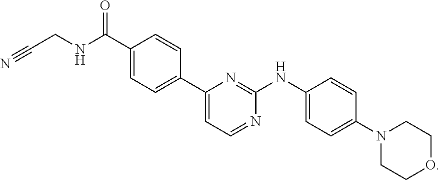 [SMILES: N#CCNC(=O)c1ccc(-c2ccnc(Nc3ccc(N4CCOCC4)cc3)n2)cc1]

6. The method of claim 5, wherein the one or more tablets contain the compound in admixture with non-toxic pharmaceutically acceptable excipients.

7. The method of claim 5, wherein the myelofibrosis is primary myelofibrosis.


SEQUENCE LISTING (16 sequences)

SEQ ID NO: 1 - protein, 300 aa, Homo sapiens
  REGION 1..300: note = MISC_FEATURE - Janus kinase 2 beginning from position 833 Genbank NP 004963
SGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 2 - protein, 295 aa, Homo sapiens
  REGION 1..295: note = MISC_FEATURE - Janus kinase 1
KNQPTEVDPTHFEKRFLKRIRDLGEGHFGKVELCRYDPEDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 3 - protein, 295 aa, Homo sapiens
  REGION 1..295: note = MISC_FEATURE - Janus kinase 3
AQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWQDRPSFSALGPQLDMLWSGSRG

SEQ ID NO: 4 - protein, 296 aa, Homo sapiens
  REGION 1..296: note = MISC_FEATURE - tyk2 protein tyrosine kinase
NRDSPAVGPTTFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHAHDYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKY

SEQ ID NO: 5 - protein, 1132 aa, Homo sapiens
  REGION 1..1132: note = MISC_FEATURE - Janus kinase 2 Genbank NP 004963
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 6 - DNA, 31 bp, synthetic construct
  misc_feature 1..31: note = primer
  source 1..31: mol_type = other DNA
acgcgtcgacggtgcctttgaagaccgggat

SEQ ID NO: 7 - DNA, 34 bp, synthetic construct
  misc_feature 1..34: note = primer
  source 1..34: mol_type = other DNA
atagtttagcggccgctcagaatgaaggtcattt

SEQ ID NO: 8 - DNA, 30 bp, synthetic construct
  misc_feature 1..30: note = primer
  source 1..30: mol_type = other DNA
ccgctcgagtatgcctgccaagaccccacg

SEQ ID NO: 9 - DNA, 30 bp, synthetic construct
  misc_feature 1..30: note = primer
  source 1..30: mol_type = other DNA
cggggtaccctatgaaaaggacagggagtg

SEQ ID NO: 10 - protein, 17 aa, synthetic construct
  REGION 1..17: note = chemically synthesized peptide substrate
  SITE 1: note = MISC_FEATURE - X = biotinylated glutamic acid
  SITE 17: note = MISC_FEATURE - X = amide-modified phenylalanine
XGPWLEEEEEAYGWMDX

SEQ ID NO: 11 - DNA, 30 bp, synthetic construct
  misc_feature 1..30: note = chemically synthesized oligonucleotide
  source 1..30: mol_type = other DNA
ggaggatcctgatctctctcgctgtgagac

SEQ ID NO: 12 - DNA, 28 bp, synthetic construct
  misc_feature 1..28: note = chemically synthesized oligonucleotide
  source 1..28: mol_type = other DNA
aggcgtcgacttcttcttcatggttctg

SEQ ID NO: 13 - DNA, 31 bp, synthetic construct; identical to SEQ ID NO: 6
  misc_feature 1..31: note = forward primer of JAK2
  source 1..31: mol_type = other DNA

SEQ ID NO: 14 - DNA, 33 bp, synthetic construct
  misc_feature 1..33: note = reverse primer of JAK2
  source 1..33: mol_type = other DNA
atagtttagcggccgctcagaatgaaggtcatt

SEQ ID NO: 15 - DNA, 35 bp, synthetic construct
  misc_feature 1..35: note = forward primer of JAK3
  source 1..35: mol_type = other DNA
gaagtcgactatgcctgccaagaccccacgatctt

SEQ ID NO: 16 - protein, 17 aa, synthetic construct
  REGION 1..17: note = chemically synthesized peptide substrate
  SITE 1: note = MISC_FEATURE - X = biotinylated glutamic acid
XQEDEPEGDYFEWLEPE